(19) United States
(12) Patent Application Publication
(10) Pub. No.: US 2024/0425900 A1
(43) Pub. Date: Dec. 26, 2024

(54) METHODS FOR DETERMINING AND/OR MONITORING THE HEALTH STATUS OF FISH

(71) Applicant: WELLFISH TECH LIMITED, Paisley (GB)

(72) Inventors: Brian M. QUINN, Paisley (GB); Josip BARISIC, Paisley (GB); Shahadate REZVY, Paisley (GB)

(21) Appl. No.: 18/687,075

(22) PCT Filed: Aug. 30, 2022
(86) PCT No.: PCT/GB2022/052209

§ 371 Date: Feb. 27, 2024

(30) Foreign Application Priority Data
Aug. 30, 2021 (GB) 2112345.0

(51) Int. Cl. C12Q 1/32; C12Q 1/40; C12Q 1/50; C12Q 1/52; G01N 33/70; G01N 33/84

(52) U.S. Cl. CPC C12Q 1/32; C12Q 1/40; C12Q 1/50; C12Q 1/52; G01N 33/70; G01N 33/84; G01N 2333/4603; G01N 2800/52

(57) ABSTRACT

The present invention relates to methods for determining the health status of populations of fish and diagnosing conditions or diseases in unhealthy fish. In particular, the present invention relates to blood biomarkers for assessing the health status and diagnosing conditions and diseases in populations of fish.

### FIELD OF THE INVENTION

The present invention relates to biomarkers and particular methods for monitoring the health status of fish populations, preferably but not limited to, farmed fish. The present invention further relates to diagnosing a condition or disease and/or monitoring the progression of the condition or disease of a population of fish.

### BACKGROUND OF THE INVENTION

Aquaculture is the fastest growing food supply sector in the world. The farming of aquatic animals grew on average 5.3% per year between 2001 and 2018. In 2018, the world's total aquaculture production reached an all-time high of 114.5 million tonnes in live weight with a total estimated first-sale value of US dollars (US$) 263 billion. Finfish accounted for 54 million tonnes with a value of $139.7 billion (FAO, 2020).

The international Salmonid industry is worth an estimated $15.4 billion (2017) with the main production sites in Norway, Chile, Scotland and Canada (FAO, 2020). The total supply of all farmed salmonids exceeded 2.2 million tonnes (gross weight) in 2018, more than double the wild catch and increased with a 7% compound annual growth rate in 2019 to just over 2.6 million tonnes (FAO, 2020).

Good fish health is of obvious importance to the salmon aquaculture industry, helping to reduce expenditure and increase productivity and ultimately profitability. Globally, mortality in salmon farming is estimated to be around 20%, greatly impacting on profitability and creating a negative public perception of the industry. Of the 48 million smolts put to sea in Scotland in 2014, 9 million (26.7%) died during the two-year production cycle, with mortality numbers reaching 10 and 11 million for 2016 and 2017 respectively (Munro L. A. & Wallace, I. S., 2017). In Norway a mortality rate of 19% (53 million salmon) in 2016 cost the industry NOK10 billion (˜£1 billion).

Currently fish health managers have no method to rapidly assess fish health and are reliant on slow, lethal, manual techniques, that can take up to 10 days to provide results. By this time the problem may have spread throughout the site, making it more difficult to treat, and potentially contributing to high mortality rates.

Accordingly, in at least some aspects the present disclosure describes a method for determining the health status of a population of fish using a rapid blood test. This disruptive technology has been developed to augment and ultimately replace reliance on lethal histological methods and enable predictive fish health forecasting using a novel data informed, pro-active healthcare model that results in increased productivity.

### BRIEF SUMMARY OF THE INVENTION

The present invention provides clinical chemistry biomarkers suitable for monitoring health status and/or diagnosing a condition or disease in a population of fish.

According to a first aspect of the invention, there is provided a method for determining the health status of a population of fish, comprising the steps of:
(a) analysing a first sample collected from at least one fish of the population of fish to determine the amount of at least one analyte selected from the group consisting of: lactate dehydrogenase; creatine kinase; creatine kinase-myocardial band (creatine kinase-MB); alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the sample to determine a test profile; and
(b) comparing the amount of the test profile with a reference profile;
   wherein a difference in the test profile as compared to the reference profile indicates the health status of the population of fish.

A population of fish may refer to at least one, at least two, at least five, at least ten, at least one hundred, at least a thousand fish. In some embodiments, a population of fish may refer to a plurality of fish. The population of fish may include fish in one or more enclosures (e.g. pens, cages or tanks). In some embodiments, the population of fish may be wild, captive or farmed fish. In some embodiments, the population of fish are salmonid, sea bass, sea bream, sturgeon, tilapia and/or carp. Preferably, the population of fish are salmon or trout.

The term ‘analyte’ or ‘biomarker’ as used interchangeably herein means any entity, particularly a chemical, biochemical or biological entity to be assessed, e.g., whose amount (e.g., concentration or mass), activity, composition, or other properties are to be detected, measured, quantified, evaluated, analysed, etc. An ‘analyte’ or a ‘biomarker’, generally refers to a qualitative and/or quantitative measurable indicator of some biological state or condition. Biomarkers are typically molecules, biological species or biological events that can be used for the detection, diagnosis, prognosis and prediction of therapeutic response of diseases.

The test profile may refer to the amount (e.g. concentration or mass) of any one or more analytes as determined in a sample. Suitably, in some embodiments the test profile refers to the amount of any one or more analytes in the first sample, second sample, third sample or any later sample.

Reference profile as used herein may refer to the amount of any analyte in a fish or a population of fish. Suitably, in some embodiments the reference profile may refer to the amount of any one or more analytes in a healthy or unhealthy fish or population of fish. In some embodiments, the reference profile may refer to a plurality of analytes in a healthy or unhealthy fish or population of fish. For example, the reference profile may include a plurality of analytes representative of a healthy population of fish or a plurality of analytes representative of an unhealthy population of fish. In some embodiments the plurality of analytes incudes at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least forty, at least fifty, at least sixty, at least seventy, at least eighty, at least ninety, at least one hundred analytes representative of a healthy or unhealthy population of fish.

In some embodiments, the reference profile may be established by obtaining more than one sample from a fish or a population of fish over a time course. Suitably, in some embodiments, the reference profile is established by sampling a fish or a population of fish weekly, bi-weekly, monthly, quarterly or annually to determine a representative amount of the at least one analyte in a fish, a population of healthy fish and/or a population of unhealthy fish. In some embodiments, the one or more samples are obtained from fish from multiple sites. Multiple sites are referred to herein, may refer to fish or a population of fish housed in different enclosures on the same sampling site (i.e. on the same fish farm or the same body of water). Alternatively, multiple sites may refer to fish or a population of fish housed in independent sampling sites (i.e. on different fish farms or in different bodies of water).

Suitably, the representative amount may be referred to as the background level of the fish or the population of fish. Suitably, background levels as used herein may refer to the amount of each analyte in a representative healthy fish or the average amount of each analyte determined from a population of healthy fish. Alternatively, background levels as used herein may refer to the amount of each analyte in a representative unhealthy fish or the average amount of each analyte determined from a population of unhealthy fish.

In some embodiments, the reference profile may be determined from a fish or a population of fish, wherein the fish or the population of fish are members of the salmonid, cichlidae, carp or acipenseridae families. In some embodiments the fish or population of fish are shellfish.

In some embodiments, the reference profile can relate to calculated background levels from any number of salmonids for the analytes investigated which includes a 12-month sampling plan from at least one control sampling site to establish background levels. In one embodiment, the reference profile can relate to calculated background levels from salmonids (n=1,525) for the biomarkers investigated which includes a 12-month sampling plan from at least one control sampling site to establish background levels.

Suitably, in some embodiments, the reference profile may be the test profile obtained for a comparative population of fish. A comparative population of fish may include a population of fish of the same or different species, a population of fish housed under similar animal husbandry conditions (e.g. parasite treatment regimens), similar population numbers per enclosure or similar environmental conditions (e.g. water temperature and time of year sample collected). Suitably, for example the reference profile may be the test profile obtained from a first sample collected from a population of fish, which acts as a baseline for comparison with subsequent samples. In some other embodiments, the reference profile may be the test profile obtained for the same or a different population of fish, e.g. at an earlier time point.

In any aspect of the invention described herein, two or more analytes may be analysed to determine the test and/or reference profile. For example 3, 4, 5, 6, 7, 8, 9, 10, 11, 12, 13, 14, 15 or more analytes. In some embodiments, the test and/or reference profile comprises analysing any two or more analytes selected from the list comprising: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status. In some embodiments all of the recited analytes may be analysed to determine the test and/or reference profile.

Suitably, in some embodiments of any aspect of the present invention, the reference profile is used to determine an analyte reference range. Suitably, such ranges can be conceptualised or represented as unhealthy, abnormal and healthy ranges for each analyte. Unhealthy may be further divided into low unhealthy which represents an analyte amount that is lower than the healthy analyte reference range and high unhealthy which represents an analyte amount that is higher than the healthy analyte reference range. Abnormal may be further divided into low abnormal which represents an analyte amount that is lower than the healthy analyte reference range but an analyte amount that is higher than the low unhealthy analyte reference range and high abnormal which represents an analyte amount that is higher than the healthy analyte reference range but an analyte amount that is lower than the high unhealthy analyte reference range. Such analyte reference ranges can be conceptualised or represented as a “traffic light” system, with healthy range indicated by green, abnormal (high and low) range indicated by amber and unhealthy (high and low) range indicated by red.

For example, in some embodiments a representative healthy reference range of lactate dehydrogenase in Salmonids is any amount of lactate dehydrogenase up to 1500 U/L (green), the high abnormal range is 2500 U/L and above (amber) and the high unhealthy range is 3300 U/L and above (red).

In some embodiments, a representative healthy reference range of creatine kinase in Salmonids is any amount of creatine kinase up to 2000 U/L (green), the high abnormal range is 5000 U/L and above (amber) and the high unhealthy range is 7000 U/L and above.

In some embodiments, a representative healthy reference range of creatine kinase-MB in Salmonids is any amount of creatine kinase-MB up to 3000 U/L (green), the high abnormal range is 8000 U/L and above (amber) and the high unhealthy range is 11000 U/L and above.

In some embodiments, a representative healthy reference range of alanine aminotransferase in Salmonids is any amount of alanine aminotransferase up to 4 U/L (green), the high abnormal range is 8 U/L and above (amber) and the high unhealthy range is 10 U/L and above.

In some embodiments, a representative healthy reference range of aspartate aminotransferase in Salmonids is any amount of aspartate aminotransferase up to 100 U/L (green), the high abnormal range is 300 U/L and above (amber) and the high unhealthy range is 400 U/L and above.

In some embodiments, a representative healthy reference range of potassium in Salmonids is an amount of potassium of 2.5 mmol/L (green), the low abnormal range is any amount of potassium of 2 mmol/L or less (amber), the high abnormal range is 3 mmol/L and above (amber), the low unhealthy range is 1 mmol/L or less (red) and the high unhealthy range is 3.25 mmol/L and above (red).

In some embodiments, a representative healthy reference range of sodium/potassium ratio in Salmonids is a sodium/potassium ratio of 50 or above (green), the low abnormal range is a ratio of 40 or less (amber), the low unhealthy range is a ratio of 20 or less (red), the high abnormal range is a ratio of 80 or above and the high unhealthy range is a ratio of 93 or above.

In some embodiments, a representative healthy reference range of lactate in Salmonids is an amount of lactate of 5 mmol/L or above (green), the low abnormal range is 1 mmol/L or less (amber), the high abnormal range is 7 mmol/L or above (amber) and the high unhealthy range is 9 mmol/L or above (red).

In some embodiments, a representative healthy reference range of amylase in Salmonids is any amount of amylase up to 700 U/L (green), the high abnormal range is 1200 U/L and above (amber), the high unhealthy range is 1700 U/L or above (red).

In some embodiments, a representative healthy reference range of creatinine in Salmonids is an amount of creatinine of 40 μmol/L and above (green), the low abnormal range is 10 μmol/L and below (amber), the high abnormal range is 65 μmol/L and above (amber), the low unhealthy range is 2 μmol/L and below (red) and the high unhealthy range is 85 μmol/L and above (red).

In some embodiments, a representative healthy reference range of total protein in Salmonids is an amount of total protein of 40 g/L and above (green), the low abnormal range is 26 g/L and below (amber), the high abnormal range is 49 g/L and above (amber), the low unhealthy range is 18 g/L and below (red) and the high unhealthy range is 60 g/L and above (red).

In some embodiments, a representative healthy reference range of phosphorous in Salmonids is a concentration of phosphorous of 5 mmol/L and above (green), the low abnormal range is 2 mmol/L and below (amber), the high abnormal range is 10 mmol/L and above (amber), the low unhealthy range is 1 mmol/L and below (red) and the high unhealthy range is 12 mmol/L and above (red).

In some embodiments, a representative healthy reference range of sodium in Salmonids is an amount of sodium of 155 mmol/L and above (green), the low abnormal range is 149 mmol/L and below (amber), the high abnormal range is 165 mmol/L and above (amber), the low unhealthy range is 144 mmol/L and below (red) and the high unhealthy range is 170 mmol/L and above (red).

In some embodiments, a representative healthy reference range of zinc in Salmonids is an amount of zinc of 250 μmol/L and above (green), the low abnormal range is 150 μmol/L and below (amber), the high abnormal range is 350 μmol/L and above (amber), the low unhealthy range is 50 μmol/L and below (red) and the high unhealthy range is 400 μmol/L and above (red).

In some embodiments, a representative healthy reference range of ammonia in Salmonids is an amount of ammonia of 1000 μmol/L and above (green), the low abnormal range is 200 μmol/L and below (amber), the high abnormal range is 1800 μmol/L and above (amber), and the high unhealthy range is 2300 μmol/L and above (red).

In some embodiments, a representative healthy reference range of alkaline phosphatase in Salmonids is an amount of alkaline phosphatase of 600 U/L and above (green), the low abnormal range is 300 U/L and below (amber), the high abnormal range is 900 U/L and above (amber), the low unhealthy range is 100 U/L and below (red) and the high unhealthy range is 1200 U/L and above (red).

In some embodiments, a representative healthy reference range of iron in Salmonids is an amount of iron of 20 μmol/L and above (green), the low abnormal range is 3 μmol/L and below (amber), the high abnormal range is 40 μmol/L and above (amber), and the high unhealthy range is 50 μmol/L and above (red).

In some embodiments, a representative healthy reference range of chloride in Salmonids is an amount of chloride of 140 mmol/L and above (green), the low abnormal range is 134 mmol/L and below (amber), the high abnormal range is 150 mmol/L and above (amber), the low unhealthy range is 130 mmol/L and below (red), and the high unhealthy range is 154 mmol/L and above (red).

In some embodiments, a representative healthy reference range of carbon dioxide in Salmonids is an amount of carbon dioxide of 8 mmol/L and above (green), the low abnormal range is 3 mmol/L and below (amber), the high abnormal range is 16 mmol/L and above (amber), the low unhealthy range is 1 mmol/L and below (red), and the high unhealthy range is 20 mmol/L and above (red).

In some embodiments, a representative healthy reference range of albumin in Salmonids is an amount of albumin of 15 g/L and above (green), the low abnormal range is 12 g/L and below (amber), the high abnormal range is 20 g/L and above (amber), the low unhealthy range is 9 g/L and below (red), and the high unhealthy range is 22 g/L and above (red).

In some embodiments, a representative healthy reference range of calcium in Salmonids is an amount of calcium of 3 mmol/L and above (green), the low abnormal range is 2 mmol/L and below (amber), and the high abnormal range is 4 mmol/L and above (amber).

In some embodiments, a representative healthy reference range of magnesium in Salmonids is a concentration of magnesium of 1.5 mmol/L and above (green), the low abnormal range is 1 mmol/L and below (amber), the high abnormal range is 2 mmol/L and above (amber), and the high unhealthy range is 3 mmol/L and above (red).

In some embodiments, a representative healthy reference range of total bilirubin in Salmonids is an amount of total bilirubin of 10 μmol/L and above (green), the low abnormal range is 2 μmol/L and below (amber), the high abnormal range is 16 μmol/L and above (amber), and the high unhealthy range is 18 μmol/L and above (red).

In some embodiments, a representative healthy reference range of globulins in Salmonids is an amount of globulins of 25 g/L and above (green), the low abnormal range is 14 g/L and below (amber), the high abnormal range is 29 g/L and above (amber), the low unhealthy range is 9 g/L and below (red), and the high unhealthy range is 38 g/L and above (red).

In some embodiments, a representative healthy reference range of total iron binding capacity in Salmonids is an amount of total iron binding capacity of 45 μmol/L and above (green), the low abnormal range is 35 μmol/L and below (amber), the high abnormal range is 55 μmol/L and above (amber), the low unhealthy range is 28 μmol/L and below (red), and the high unhealthy range is 65 μmol/L and above (red).

In some embodiments, a representative healthy reference range of copper in Salmonids is an amount of copper of 8 μmol/L and above (green), the low abnormal range is 6 μmol/L and below (amber), the high abnormal range is 12 μmol/L and above (amber), the low unhealthy range is 4 μmol/L and below (red), and the high unhealthy range is 14 μmol/L and above (red).

In some embodiments of the present invention, the healthy range, abnormal range and unhealthy range is determined by the concentration of each biomarker as indicated in Table 1. Where the value of “0” is indicated in the table, this is representative of a below threshold reading i.e. below the limit of detection of the assay. Suitably, where a value of “0” is attributed, the analyte is considered to fall in the next analyte reference range. For example, lactate dehydrogenase reads “0” for low unhealthy and low abnormal. The skilled person may interpret this as 0-1500 is a healthy reference value.

The skilled person will understand that the analyte reference ranges above apply to any aspect of the present invention as described herein. The analyte reference ranges as described above and in Table 1 are indicative of representative ranges in Salmonids. The skilled person can suitably apply equivalent ranges to other species such as cichlidae, carp or acipenseridae and shellfish. Suitably, in some embodiments, a healthy range for each analyte is determined by the background level of the reference profile as described above. Suitably, in some embodiments the background level may refer to the amount of each analyte in a representative healthy fish or the average amount of each analyte determined from a population of healthy fish. The analyte reference range can then be determined as the mean of the background level of a healthy fish or a population of fish±1 SD, with ±1 to 2 SDs representing an abnormal range indicative of a degeneration in fish health, and ±more than 2 SDs representing unhealthy samples.

TABLE 1

| | | Low | Low | | High | High |
|---|---|---|---|---|---|---|
| Analyte | Unit | Unhealthy | Abnormal | Healthy | Abnormal | Unhealthy |
| Lactate | U/L | 0 | 0 | 1500 | 2500 | 3300 |
| dehydrogenase | | | | | | |
| Creatine kinase | U/L | 0 | 0 | 2000 | 5000 | 7000 |
| Creatine kinase- | U/L | 0 | 0 | 3000 | 8000 | 11000 |
| MB | | | | | | |
| Alanine | U/L | 0 | 0 | 4 | 8 | 10 |
| aminotransferase | | | | | | |
| Aspartate | U/L | 0 | 0 | 100 | 300 | 400 |
| aminotransferase | | | | | | |
| Potassium | mmol/L | 1 | 2 | 2.5 | 3 | 3.25 |
| Sodium/potassium | ratio | 20 | 40 | 50 | 80 | 93 |
| Lactate | mmol/L | 0 | 1 | 5 | 7 | 9 |
| Amylase | U/L | 0 | 0 | 700 | 1200 | 1700 |
| Creatinine | μmol/L | 2 | 10 | 40 | 65 | 85 |
| Total protein | g/L | 18 | 26 | 40 | 49 | 60 |
| Phosphorous | mmol/L | 1 | 2 | 5 | 10 | 12 |
| Sodium | mmol/L | 144 | 149 | 155 | 165 | 170 |
| Zinc | μmol/L | 50 | 150 | 250 | 350 | 400 |
| Ammonia | μmol/L | 0 | 200 | 1000 | 1800 | 2300 |
| Alkaline | U/L | 100 | 300 | 600 | 900 | 1200 |
| phosphatase | | | | | | |
| Iron | μmol/L | 0 | 3 | 20 | 40 | 50 |
| Chloride | mmol/L | 130 | 134 | 140 | 150 | 154 |
| Carbon dioxide | mmol/L | 1 | 3 | 8 | 16 | 20 |
| Albumin | g/L | 9 | 12 | 15 | 20 | 22 |
| Calcium | mmol/L | 2 | 2 | 3 | 4 | 4 |
| Magnesium | mmol/L | 0 | 1 | 1.5 | 2 | 3 |
| Total bilirubin | μmol/L | 0 | 2 | 10 | 16 | 18 |
| Globulins | g/L | 9 | 14 | 25 | 29 | 38 |
| Total iron binding | μmol/L | 28 | 35 | 45 | 55 | 65 |
| capacity | | | | | | |
| Copper | μmol/L | 4 | 6 | 8 | 12 | 14 |

In one embodiment, the first aspect further comprises monitoring the health status of a population of fish. Accordingly, the first aspect further comprises steps:
(c) analysing at least one later sample collected from at least one fish of the population of fish to determine a later test profile;
(d) comparing the amount of the at least one analyte present in the later test profile with a reference profile; optionally wherein the reference profile is the test profile obtained from the first sample;
   wherein a difference in the amount of the at least one analyte between the later test profile with the reference profile indicates a change in the health status of the population of fish.

According to a second aspect of the present invention, there is provided a method for monitoring health status of a population of fish, comprising the steps of:
(a) analysing a first sample collected from at least one fish of the population of fish at a first time point to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile; and
(b) analysing at least a second sample collected from at least one fish of the population of fish to determine the amount of the same at least one analyte present in the at least second sample to determine a second test profile;
(c) comparing the amount of the at least one analyte present in the at least second sample with a reference profile; optionally wherein the reference profile is the test profile from the first sample;
wherein a difference in the amount of the at least one analyte between the first and/or second test profile with the reference profile indicates a change in the health status of the population of fish.

Suitably, in some embodiments, a change in the health status of the population of fish may refer to improvement or a worsening in health status. Worsening may refer to an increase or decrease in the amount of at least one analyte in the sample as compared to the reference profile. Worsening may refer to the at least one analyte entering the abnormal range or entering the unhealthy range. Improving may refer to an increase or decrease in the amount of analyte in the sample. Improving may refer to the amount of the analyte entering the abnormal range from the unhealthy range or entering the normal range from the abnormal range or unhealthy range.

The skilled person will understand that a greater number of analytes that are classified as abnormal and/or unhealthy is more strongly indicative of an unhealthy fish or a population of fish. Similarly, a greater number of analytes that are classified as abnormal and/or unhealthy is more strongly indicative that the fish or a population of fish have a disease as described herein.

Suitably, in one embodiment, at least one, at least two, at least three, at least four, at least five, at least six, at least seven, at least eight, at least nine, at least ten, at least eleven, at least twelve, at least thirteen, at least fourteen, at fifteen, at least sixteen, at least seventeen, at least eighteen, at least nineteen or at least twenty of the analytes described herein being classified in the abnormal and/or unhealthy analyte reference ranges indicates an unhealthy fish.

In one embodiment, the second aspect further comprises the step of comparing the amount of the at least one analyte present in the first test profile with a reference profile. The skilled person would understand that it may be desirable to perform the method of the first aspect to first determine the health status of a population of fish and then continue to monitor the health status of the population of fish by performing the method of the second aspect of the invention.

Suitably, there is provided a method of determining the health status of a population of fish and monitoring the health status of the population of fish, wherein the method comprises:
(a) analysing a first sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the sample to determine a test profile; and
(b) comparing the amount of the at least one analyte present in the test profile with a reference profile;
   wherein a difference in the amount of the at least one analyte in the test profile as compared to the reference profile indicates the health status of the population of fish and
(c) analysing at least a second sample collected from at least one fish of the population of fish to determine the amount of the same at least one analyte present in the at least second sample to determine a second test profile;
(d) comparing the amount of the second test profile with a reference profile; optionally wherein the reference profile is the first test profile;
wherein a difference in the amount of the at least one analyte between the first and/or second test profile with the reference profile indicates a change in the health status of the population of fish.

In some embodiments according to the first and/or second aspects, the method may be performed prior to observing physical or behavioural characteristics of a condition or disease. In one embodiment, the health status of the fish provides an early indication of the condition or disease prior to observing physical or behavioural characteristic of the condition of disease.

Physical or behavioural characteristics of a condition or disease may refer to, but are not limited to, loss of appetite, weakness, moribund, loss of balance or buoyancy control, changes to swimming patterns, separation from the group, gasping or mouthing for air, changes to respiratory rate or laboured breathing and/or clamped fins.

The inventors have surprisingly found that lactate dehydrogenase is a highly predictive analyte of an unhealthy fish status.

Suitably, in some embodiments, the method according to the first, second or third aspect, at least one analyte are any of those as defined in steps (a) or (b). In some embodiments, the method according to the first, second or third aspect comprises analysing a sample collected from said population of fish to determine the amount of lactate dehydrogenase, and optionally one or more further analytes selected from the group consisting of: creatine kinase; creatine kinase-MB; alanine aminotransferase; aspartate aminotransferase; potassium; sodium/potassium ratio; lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase and creatine kinase, and optionally one or more further analytes selected from the group consisting of: creatine kinase-MB; alanine aminotransferase; aspartate aminotransferase; potassium; sodium/potassium ratio; lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase and creatine kinase-MB, and optionally one or more further analytes selected from the group consisting of: alanine aminotransferase; aspartate aminotransferase; potassium; sodium/potassium ratio; lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase, creatine kinase-MB, and alanine aminotransferase, and optionally one or more further analytes selected from the group consisting of: aspartate aminotransferase; potassium; sodium/potassium ratio; lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase, creatine kinase-MB, alanine aminotransferase, and aspartate aminotransferase and optionally one or more further analytes selected from the group consisting of: potassium; sodium/potassium ratio; lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase and potassium and optionally one or more further analytes selected from the group consisting of: sodium/potassium ratio; lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium and sodium/potassium ratio and optionally one or more further analytes selected from the group consisting of: lactate; amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio and lactate and optionally one or more further analytes selected from the group consisting of: amylase; creatinine; total protein; phosphorous; sodium; zinc; and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate and amylase and optionally one or more further analytes selected from the group consisting of: creatinine; total protein, phosphorous, sodium, zinc, and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate, amylase and creatinine and optionally one or more further analytes selected from the group consisting of: total protein, phosphorous, sodium, zinc, and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate, amylase, creatinine and total protein optionally one or more further analytes selected from the group consisting of: phosphorous, sodium, zinc, and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate, amylase, creatinine, total protein and phosphorous, and optionally one or more further analytes selected from the group consisting of: sodium, zinc, and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate, amylase, creatinine, total protein, phosphorous and sodium, and optionally one or more further analytes selected from the group consisting of: zinc and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate, amylase, creatinine, total protein, phosphorous, sodium, zinc and optionally ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase, potassium, sodium/potassium ratio, lactate, amylase, creatinine, total protein, phosphorous, sodium, zinc and ammonia.

In some embodiments of the first, second or third aspects, the method comprises analysing a sample collected from said population of fish to determine the amount of lactate dehydrogenase, creatine kinase, creatine kinase-MB, alanine aminotransferase, aspartate aminotransferase and potassium.

Suitably, the method of the first, second or third aspects as described herein may comprise analysing a sample collected from said population of fish to determine the amount of, in any number and combination of the list comprising: lactate dehydrogenase, creatine kinase, creatine kinase-MB, alanine aminotransferase, aspartate aminotransferase, potassium, sodium/potassium ratio, lactate, amylase, creatinine, total protein, phosphorous, sodium, zinc, ammonia, alkaline phosphatase, iron; chloride, carbon dioxide, albumin, calcium, magnesium, total bilirubin, globulins, total iron binding capacity, copper, and total antioxidative status. Suitably, in some embodiments the amount of all of the recited analytes are determined.

In some embodiments, monitoring health status of a population of fish comprises repeated monitoring. In some embodiments, repeated monitoring can be regular or irregular monitoring. Regular monitoring may refer to monitoring the health status of the population of fish at routine intervals such as daily, weekly, monthly, annually. Irregular monitoring may refer to monitoring the health status of the population of fish on an ad hoc basis. Suitably, in some embodiments, continuous health status monitoring of the fish population comprises performing the method of the first aspect, second aspect and/or third aspect of the invention every week, every two weeks, every three weeks, every four weeks, ever five weeks, every six weeks. The first, second and/or third aspect of the invention may be performed monthly or annually. Suitably, it may be desirable to perform the first, second and/or third aspect of the invention in spring months and/or in summer months.

The frequency of monitoring may vary depending on the temperature of water as fish in warmer waters (e.g. water temperature greater than 10° C.) are more susceptible to pests and disease. Suitably, in some embodiments more frequent monitoring is required. In some embodiments, in warmer water the monitoring the health status of a population of fish is more frequent. Suitably, the population of fish may be monitored daily, bi-weekly, weekly and/or bi-monthly. In colder water environments, the population of fish may be monitored less frequently (e.g. water temperature less than 9° C.). Suitably, the population of fish may be monitored weekly, bi-monthly, monthly, quarterly or annually.

Suitably, in some embodiments, monitoring health status of a population of fish is repeated monitoring by obtaining at least one sample from a population of fish samples every 4 weeks during the colder water months and/or every 2 weeks in warmer water months.

In some embodiments, the method of the first, second and/or third aspect of the invention comprises analysing a sample from at least one fish from a population of fish. Suitably, the sample may refer to the first sample and/or the at least one later sample. Preferably, in some embodiments more than one sample is obtained from the population of fish. Suitably, the method of the first, second and/or third aspect of the invention comprises analysing a plurality of samples from a plurality of fish from a population of fish. Suitably, a plurality of fish may include at least one, least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand fish. Suitably, a plurality of samples may include at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand samples obtained from one or more fish from a population of fish.

In some embodiments, monitoring the health status of a population of fish comprises analysing a sample from at least one fish from a population of fish. Preferably, a sample is from a plurality of fish. In one embodiment, the method of the first, second and/or third aspect of the invention the sample is analysed from a population of fish from at least one pen, at least two pens, at least three pens, at least four pens, at least five pens, at least ten pens. Suitably, a sample is analysed from a plurality of pens. In some embodiments, a population of fish includes fish housed in different pens.

In one embodiment according the first, second and/or third aspect of the invention, at least ten fish from a population of fish from at least 3 pens are sampled every four weeks during the colder water months and every two weeks in warmer water months. Suitably, in some embodiments, during the colder water months at least 30 fish are sampled every four weeks. Suitably, in some embodiments, during the warmer water months, at least 30 fish are sampled every two weeks. Suitably, in some embodiments eighteen samples are collected per site per annum.

It is obvious to the skilled person that colder and warmer water months are variable across geographical locations and are variable year on year. In some embodiments, colder water months span November to April and warmer water months span May to October.

### Diagnosis

The present invention further relates to diagnosing a population of fish with a condition or a disease. As used herein, diagnosis or diagnosing may refer to determining, identifying or classifying a condition or disease in a population of fish. Suitably, the skilled person would understand that diagnosing a population of fish includes determining the incidence or the prevalence of a condition or disease in the population of fish. Suitably, in some embodiments of the invention as described herein, diagnosing a population of fish with a condition or a disease includes identifying an outbreak of a condition or a disease in the population of fish. Diagnosing includes identifying any disease or condition in a population of fish. This may include further classification of disease type such as pancreas disease, cardiomyopathy syndrome, complex gill disease/gill issues, heart and skeletal muscle inflammation, osmoregulation issues. Conditions or diseases may also comprise dehydration, GI loss, renal disease, shock, circulatory failure, low blood sodium, metabolic alkalosis, metabolic acidosis, chronic kidney disease, pancreatitis, renal insufficiency, malabsorption, poor diet, loss of blood, anaemia, hepatitis, cirrhosis, haemolytic diseases, obstruction of biliary, hepatic and/or pancreatic ducts, impaired kidney function, kidney disease, liver disease, gill pathology, infections, protein loss, malnutrition, malignancy, starvation, infection, immunosuppression, haemolytic anaemia, inflammation, hepatitis, drug induced liver damage, heart damage, trauma, bone disease and periods of bone growth, hypothyroidism, pernicious anaemia, muscle trauma, skeletal and cardiac muscle damage, and haemorrhage.

Accordingly, a fourth aspect of the present invention provides a method of diagnosing a population of fish with a condition or a disease, the method comprising:
(a) analysing a sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the sample to determine a test profile; and
(b) comparing the amount of the at least one analyte present in the test profile with a reference profile;
   wherein a difference in the amount of the at least one analyte in the test profile as compared to the reference profile indicates the population of fish have a condition or a disease.

As discussed, it has been found that lactate dehydrogenase is the most predictive of an unhealthy fish status, but surprisingly the inventors have found creatine-kinase MB is the most predictive analyte for diagnosing an unhealthy fish with any one of pancreas disease, cardiomyopathy syndrome, heart and skeletal muscle inflammation, gill issues and osmoregulation issues.

Suitably, in some embodiments the method comprises analysing a sample collected from said population of fish to determine the amount of creatine kinase-MB and optionally one or more further analytes selected from the group consisting of: lactate dehydrogenase, amylase, iron, creatinine, alanine aminotransferase; creatine kinase; lactate; total protein; potassium; sodium/potassium ratio; sodium; alkaline phosphatase; aspartate aminotransferase; and chloride present in the sample.

In some embodiments, the method comprises analysing a sample collected from said population of fish to determine the amount of creatine kinase-MB and lactate dehydrogenase, and optionally one or more further analytes selected from the group consisting of: amylase, iron, creatinine, alanine aminotransferase; creatine kinase; lactate; total protein; potassium; sodium/potassium ratio; sodium; alkaline phosphatase; aspartate aminotransferase; and chloride present in the sample.

In some embodiments, the method comprises analysing a sample collected from said population of fish to determine the amount of creatine kinase-MB, lactate dehydrogenase and amylase, and optionally one or more further analytes selected from the group consisting of: iron, creatinine, alanine aminotransferase; creatine kinase; lactate; total protein; potassium; sodium/potassium ratio; sodium; alkaline phosphatase; aspartate aminotransferase; and chloride present in the sample.

In some embodiments, the method comprises analysing a sample collected from said population of fish to determine the amount of creatine kinase-MB, lactate dehydrogenase and alanine aminotransferase, and optionally one or more further analytes selected from the group consisting of: amylase, iron, creatinine, creatine kinase; lactate; total protein; potassium; sodium/potassium ratio; sodium; alkaline phosphatase; aspartate aminotransferase; and chloride present in the sample.

In some embodiments, the method comprises analysing a sample collected from said population of fish to determine the amount of creatine kinase-MB, lactate dehydrogenase and lactate, and optionally one or more further analytes selected from the group consisting of: alanine aminotransferase, amylase, iron, creatinine, creatine kinase, total protein, potassium, sodium/potassium ratio, sodium, alkaline phosphatase, aspartate aminotransferase, and chloride present in the sample.

In some embodiments, the method comprises analysing a sample collected from said population of fish to determine the amount of creatine kinase-MB, lactate dehydrogenase, alanine aminotransferase, and lactate and optionally one or more further analytes selected from the group consisting of: amylase, iron, creatinine, creatine kinase, total protein, potassium, sodium/potassium ratio, sodium, alkaline phosphatase, aspartate aminotransferase, and chloride present in the sample.

In some embodiments, the method comprises analysing a sample collected from said population of fish to determine the amount of creatine kinase-MB, lactate dehydrogenase, alanine aminotransferase and creatine kinase, and optionally one or more further analytes selected from the group consisting of: amylase, iron, creatinine, lactate, total protein, potassium, sodium/potassium ratio, sodium, alkaline phosphatase, aspartate aminotransferase, and chloride present in the sample.

In some embodiments, the method comprises analysing a sample collected from said population of fish to determine the amount of creatine kinase-MB and creatine kinase, and optionally one or more further analytes selected from the group consisting of: total protein, iron, lactate dehydrogenase, amylase, alanine aminotransferase, aspartate aminotransferase, chloride, sodium/potassium ratio, sodium, lactate, potassium, creatinine and alkaline phosphatase present in the sample.

In some embodiments, the method comprises analysing a sample collected from said population of fish to determine the amount of creatine kinase-MB, creatine kinase and total protein, and optionally one or more further analytes selected from the group consisting of: iron, lactate dehydrogenase, amylase, alanine aminotransferase, aspartate aminotransferase, chloride, sodium/potassium ratio, sodium, lactate, potassium, creatinine and alkaline phosphatase present in the sample.

In some embodiments, the method comprises analysing a sample collected from said population of fish to determine the amount of any combination and any number of analytes selected from the list comprising: creatine kinase-MB, lactate dehydrogenase, lactate, alanine aminotransferase, amylase, iron, creatinine, creatine kinase, total protein, potassium, sodium/potassium ratio, sodium, alkaline phosphatase, aspartate aminotransferase, and chloride present in the sample.

In some embodiments, the method comprises analysing a sample collected from said population of fish to determine the amount of creatine kinase-MB, lactate dehydrogenase, alanine aminotransferase, amylase, creatinine and iron present in the sample.

In some embodiments, the method comprises analysing a sample collected from said population of fish to determine the amount of creatine kinase-MB, lactate dehydrogenase, lactate, creatine kinase, aspartate aminotransferase and alanine aminotransferase present in the sample.

In some embodiments, the method comprises analysing a sample collected from said population of fish to determine the amount of creatine kinase-MB, creatine kinase, lactate dehydrogenase, alanine aminotransferase, iron, and aspartate aminotransferase.

In one embodiment, an increase in creatine kinase-MB, lactate dehydrogenase and alanine aminotransferase, and a decrease in amylase, creatinine and iron indicates the population of fish have pancreas disease.

In one embodiment an increase in creatine kinase-MB, lactate dehydrogenase, lactate, creatine kinase and aspartate aminotransferase, and a decrease in alanine aminotransferase indicates the population of fish have cardiomyopathy syndrome.

In one embodiment an increase in creatine kinase-MB, lactate, lactate dehydrogenase, alanine aminotransferase, creatine kinase and aspartate aminotransferase indicates the population of fish have compromised gills.

In one embodiment an increase in creatine kinase-MB, creatine kinase, lactate dehydrogenase, alanine aminotransferase, and iron, and a decrease in aspartate aminotransferase indicates the population of fish have heart and skeletal muscle inflammation.

In some embodiments, wherein the amount of any one or more, any two or more, any three or more, any four or more, any five or more of creatine kinase-MB, lactate dehydrogenase, alanine aminotransferase, amylase, creatinine and iron are in the abnormal and/or unhealthy analyte reference range as described above, this is indicative the population of fish have pancreas disease.

Suitably, in one embodiment, wherein the amount of creatine kinase-MB, lactate dehydrogenase, alanine aminotransferase, amylase, creatinine and iron is in the abnormal and/or unhealthy analyte reference range, this is indicative the population of fish have pancreas disease.

In some embodiments, wherein the amount of any one or more, any two or more, any three or more, any four or more, any five or more of creatine kinase-MB lactate dehydrogenase, lactate, creatine kinase, aspartate aminotransferase and alanine aminotransferase are in the abnormal and/or unhealthy analyte reference range, this is indicative the population of fish have cardiomyopathy syndrome.

Suitably, in one embodiment, wherein the amount of creatine kinase-MB lactate dehydrogenase, lactate, creatine kinase, aspartate aminotransferase and alanine aminotransferase are in the abnormal and/or unhealthy analyte reference range, this is indicative the population of fish have cardiomyopathy syndrome.

In some embodiments, wherein the amount of any one or more, any two or more, any three or more, any four or more, any five or more of creatine kinase-MB, lactate, lactate dehydrogenase, alanine aminotransferase, creatine kinase and aspartate aminotransferase are in the abnormal and/or unhealthy analyte reference range, this is indicative the population of fish have compromised gills.

Suitably, in one embodiment, wherein the amount of creatine kinase-MB, lactate, lactate dehydrogenase, alanine aminotransferase, creatine kinase and aspartate aminotransferase are in the abnormal and/or unhealthy analyte reference range, this is indicative the population of fish have compromised gills.

In some embodiments, wherein the amount of any one or more, any two or more, any three or more, any four or more, any five or more of creatine kinase-MB, creatine kinase, lactate dehydrogenase, alanine aminotransferase, iron and aspartate aminotransferase are in the abnormal and/or unhealthy analyte reference range, this is indicative the population of fish have heart and skeletal muscle inflammation.

Suitably, in one embodiment, wherein the amount of creatine kinase-MB, creatine kinase, lactate dehydrogenase, alanine aminotransferase, iron and aspartate aminotransferase are in the abnormal and/or unhealthy analyte reference range, this is indicative the population of fish have heart and skeletal muscle inflammation.

It will be understood by the skilled person that the greater the number of analytes that are classified as abnormal and/or unhealthy is more strongly indicative that the fish or a population of fish have a disease as described herein.

According to a fifth aspect, there is provided herein a method for monitoring progression of a condition or disease in a population of fish, comprising the steps of:
(a) analysing a first sample collected from at least one fish of the population of fish at a first time point to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile; and
(b) analysing at least one later sample collected from at least one fish at least one later time point to determine the amount of the same at least one analyte present in the least one later sample to determine at least one later test profile;
(c) comparing the amount of the at least one analyte present in the at least one later test profile with a reference profile; optionally wherein the reference profile is the first test profile;
   wherein a difference in the amount of the at least one analyte between the at least one later test profile with the reference profile indicates progression in the condition or disease.

In one embodiment, the fifth aspect further comprises an optional step after (a) of comparing the amount of the at least one analyte present in the first test profile with a reference profile.

Progression as used herein may refer to improvement or a worsening in the condition or disease. Worsening may refer to an increase or decrease in the amount of analyte in the sample as compared to the reference profile. Worsening may refer to the analyte entering the abnormal analyte reference range as described herein or entering the unhealthy analyte reference range as described herein. In some embodiments worsening may refer to the analyte entering the abnormal range defined as ±1 to 2 SDs from background levels or entering the unhealthy range defined as ±greater than 2 SDs from background levels. Improving may refer to an increase or decrease in the amount of analyte in the sample. Improving may refer to the analyte entering the abnormal analyte reference range from the unhealthy analyte reference range as described herein or entering the healthy analyte reference range as described herein. In some embodiments, improving may refer to the amount of the analyte entering the abnormal range as defined as mean±1 to 2 SDs from the unhealthy range as defined as mean±greater than 2 SDs or entering the normal range as defined as mean±1 SD from the abnormal range or unhealthy range.

Current diagnostic testing is typically sporadic, only occurring when a specific health challenge arises. Advantageously, the present invention provides methods for both diagnostic testing before any physiological or behavioural aspects of a condition or disease are observed and when the population of fish display symptoms of a condition or disease. This has the advantage over current methods available in the field that conditions or diseases can be diagnosed earlier. Suitably, in some aspects the sample is analysed for one or more analytes comprised in Table 2. In one aspect, the one or more analyte analysed in the sample is selected based on the observed condition of the one or more impacted fish. Suitably, in some embodiments, the symptoms of a particular condition or disease as displayed by the fish may dictate the panel of biomarkers selected for analysis.

According to some embodiments of any of the aspects as described herein, the at least one analyte does not include total anti-oxidative status.

TABLE 2
List of preferred clinical chemistry assays.

| Gill & Osmoregulatory | | Cardiac & Muscle | | Liver, Kidney & | |
|---|---|---|---|---|---|
| Function | | Profile | | Pancreatic Function | |
| 1. | Sodium (Na) | 1. | Creatine kinase (CK) | 1. | Albumin (ALB) |
| 2. | Potassium (K) | 2. | Creatine kinase-myocardial | 2. | Total Protein (TP) |
| 3. | Chloride (Cl) | | band (CK-MB) | 3. | Alkaline Phosphatase |
| 4. | Carbon Dioxide | 3. | Lactate dehydrogenase (LDH) | | (ALP) |
| | (CO2) | 4. | Aspartate Aminotransferase | 4. | Alanine aminotransferase |
| 5. | Ammonia (AMM) | | (AST) | | (ALT) |
| 6. | Phosphorus (P) | 5. | Lactate (LACTA) | 5. | Total Bilirubin (TBIL) |
| | | 6. | Glucose (GLU) | 6. | Creatinine (CREA) |
| | | | | 7. | Calcium (Ca) |
| | | | | 8. | Phosphorus (P) |
| | | | | 9. | Amylase (AMY) |
| | | | | 10. | Lipase (LIP) |
| | | | | 11. | Uric Acid (URIC) |
| | | | | 12. | Butyryl Cholinesterase |
| | | | | | (BChE) |

| | |
|---|---|
| Mineral Profile | Lipid Profile |

| | | | |
|---|---|---|---|
| 1. | Phosphorus (P) | 1. | Total Cholesterol (TC) |
| 2. | Magnesium (Mg) | 2. | LDL Cholesterol (LDL) |
| 3. | Iron (Fe) | 3. | HDL Cholesterol (HDL) |
| 4. | Zinc (Zn) | 4. | Triglycerides (TG) |
| 5. | Potassium (K) | | |

In any of the aspects and embodiments discussed herein, the amount of the at least one analyte in the test profile is compared to the reference profile as described above.

In some embodiments, the reference profile is based upon healthy samples taken during routine monitoring sampling. In some other embodiments, the reference profile may be the test profile. Suitably, the reference profile may be the test profile obtained from a first sample collected from a population of fish, which acts as a baseline for comparison with subsequent samples.

In some embodiments, the reference profile is the amount of the at least one analyte in a sample obtained from at least one fish of the population of fish not displaying any behavioural or physical characteristics of a condition or disease. In some embodiments the reference profile is the test profile of a first or subsequent sample form a population of fish which is used to establish a baseline.

### Therapeutic Interventions

In one aspect of the present invention, there is provided a method according to any other aspects and embodiments wherein the method further comprises administering an effective amount of a therapeutic agent to the population of fish to treat the change in health status or the condition or disease.

The term ‘treatment’ or ‘treating’ as used herein may refer to reducing, ameliorating or eliminating one or more signs, symptoms, or effects of a disease or condition. ‘Treatment’ as used herein includes any treatment of a disease in a fish or a population of fish, and includes: (a) preventing the disease from occurring in a fish or a population of fish predisposed to the disease or at risk of acquiring the disease but has not yet been diagnosed as having it; (b) inhibiting the disease, i.e., arresting its development; (c) relieving the disease, i.e., causing regression of the disease; and (d) alleviating or reducing any symptoms of the disease.

In a further aspect, there is provided a method of treating a condition or disease in a population of fish identified by any of the first, second or third aspects as having a change in health status or any of the fourth or fifth aspects as having a condition or disease

In some embodiments, the disease or condition is pancreas disease, cardiomyopathy syndrome, gill issues/disease (including but not restricted to the specific amoebic gill disease (AGD), parasitic gill disease, viral gill disease, bacterial gill disease, zooplankton (cnidarian nematocyst)-associated gill disease, harmful algal gill disease and chemical/toxin-associated gill disease and the less specific complex gill disease (CGD)), heart and skeletal muscle inflammation and/or osmoregulatory issues. Conditions or disease may also include, but are not limited to dehydration, GI loss, renal disease, shock, circulatory failure, low blood sodium, metabolic alkalosis, metabolic acidosis, chronic kidney disease, pancreatitis, renal insufficiency, malabsorption, poor diet, loss of blood, anaemia, hepatitis, cirrhosis, haemolytic diseases, obstruction of biliary, hepatic and/or pancreatic ducts, impaired kidney function, kidney disease, liver disease, gill pathology, infections, protein loss, malnutrition, malignancy, starvation, infection, immunosuppression, haemolytic anaemia, inflammation, hepatitis, drug induced liver damage, heart damage, trauma, bone disease and periods of bone growth, hypothyroidism, pernicious anaemia, muscle trauma, skeletal and cardiac muscle damage, and haemorrhage.

Fish welfare has become increasingly important in aquaculture and continuous health monitoring during sea lice treatments is essential to ensure fish wellbeing. Clinical chemistry analysis has been previously undertaken in salmonids (Hille, S. A, 1982) and has proven a useful tool to analyse the health status of fish following pollution exposure (Bernet, D et al., 2000), feed trials (Ferri, J. et al 2011; Adel, M. et al, 2015), disease monitoring and diagnosis (Řehulka, J. 2003; Floyd-Rump, T. P. et al., 2017), toxicological studies (Steinbach, C. et al. 2014; Javed, M. et al., 2017) and to investigate pathophysiology (Benfey, T. J. & Biron, M., 2000).

It is preferable in some circumstances to not treat or to delay treatment of a population of fish classified as ‘unhealthy’ or having been diagnosed with a condition or disease with routine husbandry treatments including anti-parasite treatments such as anti-sea lice treatments, antibiotics or hydrogen peroxide. Anti-parasite treatments include but are not limited to levamisole, metronidazole or praziquantel. In some circumstances routine treatments administered to unhealthy fish populations increases the mortality rate. It is preferable in some circumstances to only treat healthy fish with routine husbandry treatments to reduce overall mortality.

Accordingly, the present invention has the advantage of determining the health status of a population of fish before treating with routine husbandry treatments. This may reduce the mortality rate of farmed fish. This also has the advantage of reducing the amount unnecessary antibiotics, anti-parasitic drugs and chemical agents entering the aquatic ecosystem.

Suitably, according to one aspect of the present invention there is provided a method for determining whether or not to treat a population of fish, or determining whether a proposed treatment is appropriate, comprising the steps of:
(a) analysing a sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile;
(b) comparing the amount of the at least one analyte present in the test profile with a reference profile;
wherein a difference in the amount of the at least one analyte between the test profile and the reference profile indicates the population of fish have developed a condition or a disease and that treatment is undesirable or an alternative treatment method is recommended.

According to another aspect of the present invention there is provided a method for determining whether or not to treat a population of fish, or determining whether a proposed treatment is appropriate, comprising the steps of:
(a) analysing a sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile;
(b) comparing the amount of the at least one analyte present in the test profile with a reference profile;
wherein a difference in the amount of the at least one analyte between the test profile and the reference profile indicates the population of fish have a change in health status;
wherein when the fish is determined to be unhealthy, treatment is undesirable or an alternative treatment method may be recommended; alternatively,
wherein when the fish is determined to be healthy, the proposed treatment may proceed.

In some embodiments, the health status may indicate a suitable therapeutic window for a proposed treatment.

In some embodiments, a proposed treatment may include any routine animal husbandry treatments such as treatment for parasitic, bacterial, amoebic and/or viral infections.

In some embodiments, the population of fish should not be treated for a parasite infection. In a preferred embodiment, the population of fish should not be treated for a sea lice infection or an alternative treatment method recommended. Parasites include but are not limited to ectoparasites such as sea lice or salmon fluke (Gyrodactylus salaris), endoparasites such as Kudoa thyrsites. Suitably, in some embodiments, the population of fish should not be treated with anti-parasitic treatments, preferably the population of fish should not be treated with anti-sea lice treatments. In some embodiments, the population of fish should not be treated with any one of levamisole, metronidazole or praziquantel.

In further embodiments, the population of fish should not be treated for a bacterial, amoebic and/or viral infection.

Suitably, in some embodiments, an alternative treatment method is providing a therapeutic agent to treat any of pancreas disease, cardiomyopathy syndrome, heart and skeletal muscle inflammation, gill disease and osmoregulatory issues. Suitably, in some embodiments, the population of fish is first treated for any of pancreas disease, cardiomyopathy syndrome, heart and skeletal muscle inflammation, gill disease and osmoregulatory issues, then the population of fish is treated with routine animal husbandry treatments.

Suitably, in some embodiments, the population of fish should not be treated and the population of fish should be harvested.

In any aspects and embodiments discussed herein, the health status and/or diagnosis of a condition or disease may indicate that a population or a sub-population of fish should be harvested. In some aspects and embodiments, the health status and/or diagnosis of a condition or disease may indicate that a population of fish require reduced feeding, a convalescence diet or early harvesting.

Harvesting as used herein may refer to slaughtering the population of fish or a sub-population of fish or individual fish. The sub-population of fish selected for harvesting may be the weakest or smallest fish. Harvesting includes any of stunning, killing and further processing of fish. Further processing of fish may include preparing the fish for human or animal consumption.

Early harvesting refers to slaughtering of fish earlier than the full two-year production cycle. Suitably, early harvesting refers to slaughtering of fish before the fish have reached their full size and weight.

### Samples

The term ‘sample’ may refer to any biofluid. In some aspects of the present invention the sample is blood or a blood fraction, such as serum or plasma. Suitably the blood or blood fraction sample is from circulating blood. A ‘later sample’ as used herein may refer to any sample collected from any one of the population of fish after the first sample. A later sample may include a first, second, third, fourth, fifth, sixth, seventh, eighth, ninth or tenth sample, collected after the first sample.

According to any one of the aspects described herein, a sample may be obtained from at least one fish from a population of fish. The skilled person would understand that ‘a sample’ as referred to herein may mean a sample obtained from one individual fish and/or samples obtained from more than one individual fish. Suitably, according to any suitable aspect of the invention as described herein, analysing a sample may include analysing a plurality of samples from a plurality of fish from a population of fish. Suitably, a plurality of fish may include at least one, least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand fish. Suitably, a plurality of samples may include at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand samples obtained from one or more fish from a population of fish.

In some embodiments, samples are collected from at least one enclosure (e.g. cage, pen or tank) per site. Preferably, samples are collected from at least two, at least three, at least four, at least five, at least six, at least seven, at least eight, at least nine or at least ten enclosures per site.

In some preferred embodiments, samples are collected from ten fish from three individual enclosures per site.

Suitably the population of fish are wild, captive or farmed fish. Captive fish include domesticated fish kept in lakes, ponds, tanks and aquariums. In some embodiments, the fish are from the salmonid, cichlidae, carp or acipenseridae families. In some embodiments the population of fish are shellfish.

In some embodiments the population of fish are any one of salmon, brown trout, rainbow trout, tilapia, carp, minnows, sea bass, sea bream and/or sea bream.

### Kit

In one aspect, there is provided a kit for use in any of the aspects and embodiments of the present invention, wherein the kit comprises one or more reagents for determining the amount of the at least one analyte in a sample, and instructions for use.

In some embodiments, the kit comprises instructions for sample collection and processing. Suitably, the instructions for sample collection and processing comprise the steps of:
Anaesthetising a fish using MS-222 following manufacturer's instructions;
Extracting whole blood from caudal vein, behind caudal fin using 21G (40 mm) needle and 2 ml syringe (new syringe/needle per fish);
Pulling and replacing the syringe plunger to release the pressure before use;
Inserting the needle behind the caudal fin, towards fish spine and stopping when resistance is felt;
Gently pulling the plunger and watching the syringe fill with blood;
Collecting 1.5 ml (minimum) to 1.6 ml (maximum) whole blood;
Placing cap on needle and dispose of needle & syringe;
Removing the needle (important to prevent haemolysis) and aspirating blood into micro tube.
Inverting the tube x3 (do not shake) to mix clotting activator and leave standing upright for a minimum of 30 min before centrifugation (Max time 4 h);
Centrifuging at 10,000 g for 5 min;
Pipetting serum into labelled tube (ensuring not to disturb the blood clot).
Ensuring tubes containing the serum are closed properly and place into plastic bag.
Cold store (4-8° C.) until ready for transportation. If freezing place in freezer as soon as possible.
Fill in details on Sample Submission Form and include with samples.

In a further embodiment, the kit comprises thermal postal pockets and a freezer pack for transportation of the samples.

### BRIEF DESCRIPTION OF THE FIGURES

FIG. 1: Schematic diagram comparing the current reactive model for fish health care with the present invention pro-active healthcare model. The current reactive model: (1) following observation of moribund fish (a) and a vet inspection (b), 3-5 fish are killed (c), for tissue biopsies (5 mm) (d) analysed by histology (e) with reports (f) taking up to 10 days. In comparison, the novel pro-active healthcare model: (2) involves continuous (weekly or bi-weekly) (a) blood sampling of representative (n=30) numbers (b) with serum (c) analysed using automated clinical chemistry technologies (d) to produce an intuitive site-specific report via a mobile platform (e) and company-wide (f) report within 24 hours facilitating data informed husbandry.

FIG. 2: Schematic diagram showing the predictive nature of the novel pro-active healthcare model of the present invention that results in increased treatment efficacy and a lower treatment cost.

FIG. 3: Pancreas disease (PD). Fish collected at four sampling points, with heathy fish (from 4 cages, n=10 per cage) compared against PD positive and PD recovered fish (8 cages, n=10 per cage). Biomarkers that showed an increased expression in PD fish included Creatine kinase-MB (CK MB) and Lactate dehydrogenase (LDH) and alanine aminotransferase (ALT). Biomarkers with decreased expression in PD infected fish include Amylase (Amy), Creatinine (Crea) and Iron (Fe).

FIG. 4: Cardiomyopathy syndrome (CMS). Fish collected from two sampling points with healthy fish (4 cages, n=10 per cage) compared against CMS positive fish (8 cages, n=10 per cage). Biomarkers that showed an increased expression in CMS infected fish include Creatine kinase-MB (CK MB), Lactate dehydrogenase (LDH), Lactate (LACTA), Creatine kinase (CK) and Aspartate aminotransferase (AST). Biomarkers with decreased expression in CMS infected fish include Alanine aminotransferase (ALT).

FIG. 5: Gill issues. Fish collected from one sampling point with healthy fish (4 cages, n=10 per cage) compared against fish with confirmed gill issues (4 cages, n=10 per cage). Biomarkers that showed an increased expression in fish with compromised gills include Creatine kinase-MB (CK MB), Lactate dehydrogenase (LDH), Lactate (LACTA), Alanine aminotransferase (ALT), Creatine kinase (CK), and Aspartate aminotransferase (AST).

FIG. 6: Heart and skeletal muscle inflammation (HMSI). Fish collected from 4 sampling points with healthy fish (3 cages, n=10 per cage) compared against HSMI confirmed fish (3 cages, n=10 per cage). Biomarkers that showed an increased expression in HSMI confirmed fish include Creatine kinase-MB (CK-MB), Lactate dehydrogenase (LDH), Alanine aminotransferase (ALT), Creatine kinase (CK), and Iron (Fe). Biomarkers with decreased expression in CMS infected fish include Aspartate aminotransferase (AST).

FIG. 7: Lactate dehydrogenase (LDH), creatine kinase-MB (CK MB), alanine aminotransferase (ALT), aspartate aminotransferase (AST) and phosphorous (P) background levels in aquaculture reared salmonids. Grey=±1 SD; light grey=±1-2 SD; dark grey=±>2 SD.

FIG. 8: Schematic diagram showing the artificial intelligence approach for fish health monitoring.

FIG. 9: The effect of 15 biomarkers on model performance. Performance decrease effect in terms of class-wise accuracy and recall associated with each of the biomarkers is shown in these feature importance diagram. The error bar is produced with five permutations of the features. Some of the biomarkers at the bottom has low impact in the two-class Healthy-unhealthy model, the have significant impact on the multi-class disease model.

FIG. 10: ROC-AUC Score and Precision-AUC score is >97% in the final model.

FIG. 11: Model produces class labels with associated class-wise probability/confidence value for its decision.

FIG. 12: Model driven fish health measuring scale using force plots

FIG. 13: Model driven fish health measuring scale using biomarker contribution plot

FIG. 14: Schematic example of a Random Forest classification. X denotes a test observation, n denotes the number of decision trees in the Random Forest, and Class z denotes the classification given by the decision tree z. The final class information of x is determined by counting the most votes.

FIG. 15: Multi-class disease model comprising of disease specific sub-models stacked to provide boosted performance.

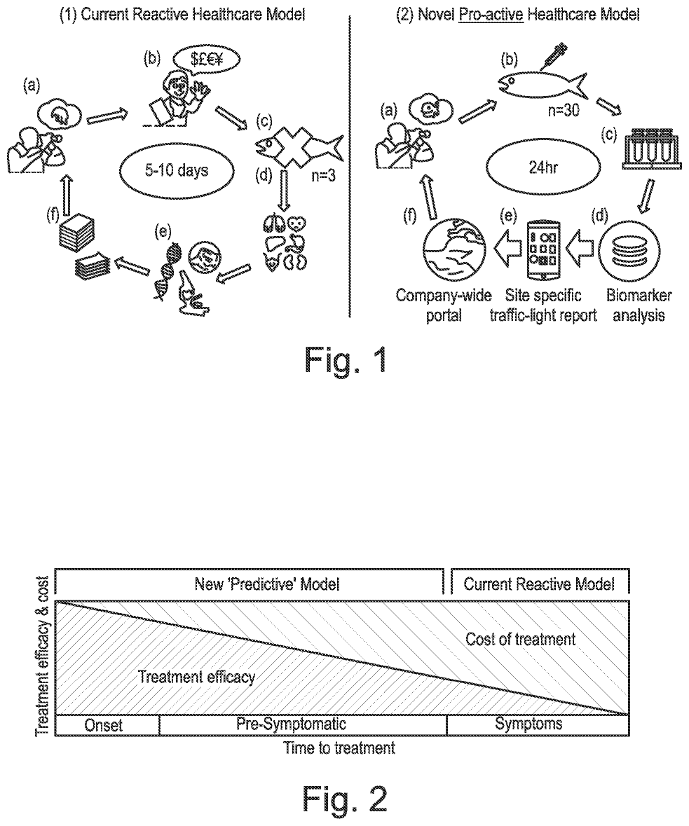

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.-in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

### Definitions

As referred to herein, the ‘difference’ in the test profile or the ‘difference’ in the at least one analyte as compared to the reference profile may refer to any positive or negative deviation from the reference value. The difference may refer to an increase in the concentration of at least one analyte when compared to the reference profile. The difference may refer to a decrease in the concentration of at least one analyte when compared to the reference profile. It will be clear to the skilled person that where more than one analyte is measured, the difference may refer to an increase of at least one analyte compared to the reference profile and a decrease of one or more different analytes when compared to the reference value. A difference may also be determined by deviation from the mean reference value. Suitably, a difference may refer to mean of the reference value±1 SD, ±2 SD, ±3 SD or ±4 SD.

The term ‘therapeutic window’ as referred to herein may refer to the optimum time to treat the population of fish with a suitable therapy. The optimum time may refer to the timeframe when the treatment is most effective and when the risk of mortality is low.

The term ‘creatine kinase-myocardial band’ may be used interchangeably with ‘creatine kinase-MB’ or ‘CK-MB’. Creatine kinase-MB refers to an isoform of creatine kinase that is predominantly, but not exclusively, expressed in heart muscles.

### Introduction

Clinical biochemistry is the cornerstone of human and veterinary medicine, used to measure the health status of organisms. Clinical biochemistry is the analysis of concentrations of numerous proteins, metabolites, enzymes and electrolytes in bodily fluids, most commonly blood-derived serum or plasma, for non-destructive diagnosis and monitoring of disease. Clinical biochemistry is a vital diagnostic tool, but despite occasional studies showing its usefulness in monitoring health status in Atlantic salmon (Salmo salar L.), it has not yet been widely utilized within the aquaculture industry based on (i) lack of established background (normal) levels and (ii) lack of clinically significance data. The inventors have generated a significant dataset to overcome both of these issues.

Biochemistry endpoints previously measured in salmonid species (Sandnes & Waagbo, 1988; Rehulka, 2003; Quinn et al., 2015; Braceland et al., 2017; Barisic et al., 2019). But there is no information available on the clinical significance of this approach to assess fish health in aquaculture. The present invention has taken existing human medical medium/high throughput clinical chemistry analysers with the software open to make the necessary changes to the settings to make the normal, medical kits used for these instruments fall within the reactive ranges for fish blood, established through research.

With a significant amount of data (33 clinical chemistry endpoints on ˜5000 fish serum samples) an AI model has been developed to aid with data interpretation and to categorise the fish as health v unhealthy (model 0) and for the unhealthy fish to further categorise the specific health challenge based on the biomarker expression. Data are also clinically interpreted through the comparison of biomarker expression between fish suffering from different health challenges.

Current methods for routine fish health assessment and disease identification in aquaculture rely on the use of lethal techniques such as tissue specific PCR and histopathology. The present invention provides methods for assessing and monitoring the health status of a population of fish and diagnosing a population of fish as having a condition or disease. The present invention has the advantage of using non-lethal, blood-based methods that are rapidly assessed using automated, medium/high throughput clinical chemistry instrumentation.

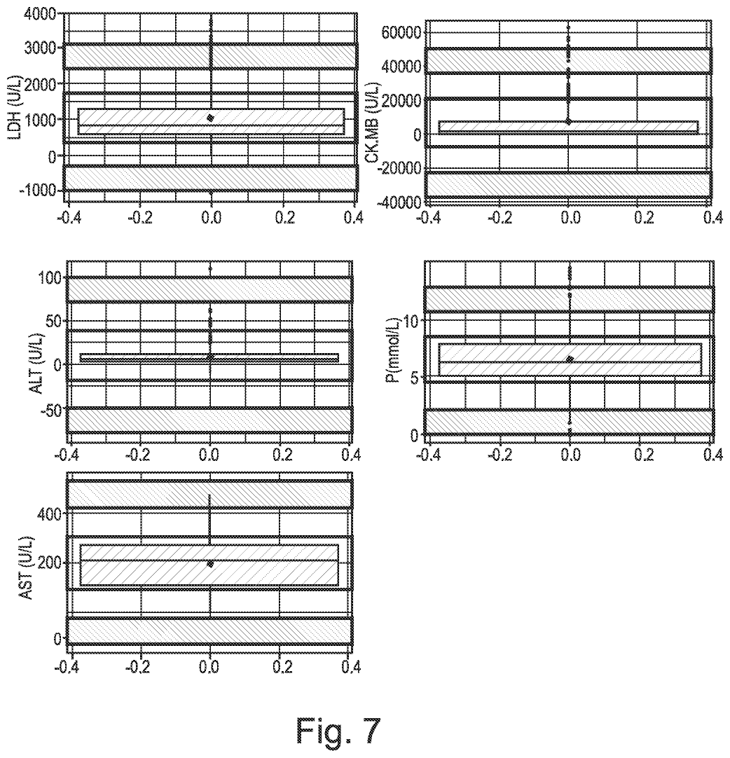

Results are interpreted against an extensive background database to enable clinical interpretation and the establishment of normal background ranges. Results are presented via a traffic light system with green indicating within the normal ranges, yellow/amber indicating between 1-2 standard deviations outside of the normal range and red begin >2 SD from the mean (see FIG. 7). An AI model has been developed to differentiate the fish into healthy or unhealthy based on their biomarker expression. Health challenge identification is undertaken using our AI model based on patterns of biomarker expression.

Using this non-lethal based approach health monitoring of a population of fish from several pens at a site can be undertaken routinely in larger sample numbers providing an overview of the health of the population. The present invention provides a practical method for continuous fish health assessment, that is rapid, non-lethal, and a blood-based method to assess fish health, similar to human and veterinary medicine to augment and ultimately replace the existing slow, lethal histology methods.

Such an approach can be applied to salmon and other commercially important fish (e.g. sea bass, sea bream, sturgeon) and invertebrate (e.g. shrimp, lobster) aquaculture markets.

The problem faced by the aquaculture industry for the assessment of fish health is the dependence on slow (5-10 days), lethal, histology-based methods. The present invention provides a re-purposed human high throughput medical technology for use on fish blood to enable rapid clinical chemistry centred health assessment, based on the continuous sampling of fish stock, similar to that used in all other livestock based agriculture. The skilled person will understand that it is not an insignificant challenge to apply continuous sampling to fish stocks, due to a lack of reference data for clinical comparison and blood sample collection difficulties. These challenges have been largely overcome by the present invention.

The advantages of the present invention in some embodiments include:
Rapid results within 24 hours
Enabling fish health managers to make data-informed husbandry decisions, facilitating predictive health forecasting, reducing mortality and increasing productivity.
Enabling the development of a pro-active fish healthcare model, augmenting the understanding of many health issues and ultimately replacing the need for histological-based analysis.

On-going and regular analysis of blood biomarkers will support existing work designed to continually improve fish welfare in commercial farming. By creating a standardised dataset and algorithm-based AI model to generate “early-warning” health indicators via a site-specific online platform within 24 hours of sample delivery, vets and fish health managers will be able to detect fish health issues earlier, thus increasing the likelihood of effective intervention by treatment, reduced feeding, convalescence diet or early harvest.

As this technique is non-lethal and automated, larger, more representative sample numbers can be analysed. Other advantages include:
The ability to assess the health of the whole fish (homeostasis), as well as the functioning of specific tissues (e.g. liver, kidney).
The cost-effective analysis of large sample numbers reduces the chances of a critical organ pathology being overlooked.
Data presentation via a traffic light system (green=healthy; orange=potential health issue; red=serious health issue requiring immediate attention).
Enables fish health managers to make data informed husbandry decisions, improving fish health, productivity and profitability.

This technique is enabled by altering the high throughput clinical chemistry instruments and biomarker reactive ranges to make them fish specific. The clinical chemistry biomarker results can then be interpreted through a substantial chemistry database for 33 biomarkers measured in thousands of salmon and trout samples, (including a 12-month sampling plan from ‘control’ sites to establish background levels). Additionally, machine Learning models/tools/algorithms identify fish as healthy or unhealthy with specific disease identification within 24 hours, based on biomarker expression allowing early treatment based on clinical chemistry expression.

Regular monthly or bi-weekly blood samples from a large cohort of fish (˜30) per site for continuous health monitoring with a report based on the “traffic light” system available within 24 hours via an online portal.

Samples can be collected for sporadic weekly/biweekly diagnostic testing to help identify a specific health challenge.

This technology can be used for, but not restricted to, continuous health monitoring for numerous biomarkers (approximately 20-30) measured monthly from each site, followed by more focused diagnostic testing (more frequent (weekly/biweekly) sampling focused on a smaller number (8-10) of biomarkers. The clinical biomarkers can be tailored in panels relating to the specific health challenge to be investigated.

### Materials and Methods:

### Sample Collection & Processing Protocol

### Materials:

2 ml syringe and 21 G needle (one per fish)
Sarstedt serum Micro tube 1.3 ml, with push cap (containing Clotting Activator; Prod Code: 41.1501.005).
2 ml Eppendorf tubes (numbered to ID individual fish).
Centrifuge.
Sample submission form

### Method:

Fish anaesthetised using MS-222 following manufacturer's instructions.
Whole blood taken from caudal vein, behind caudal fin using 21G (40 mm) needle and 2 ml syringe (new syringe/needle per fish).
Pull and replace the syringe plunger to release the pressure before use.
Insert the needle behind the caudal fin, towards fish spine. Stop when feel resistance.
Gently pull the plunger, watching the syringe fill with blood.
Collect 1.5 ml (minimum) to 1.6 ml (maximum) whole blood.
Place cap on needle and dispose of needle & syringe.
Remove needle (important to prevent haemolysis) and aspirate blood into micro tube.
Invert tube x3 (do not shake) to mix clotting activator and leave standing upright for a minimum of 30 min before centrifugation (Max time 4 h).
Centrifuge at 10,000 g for 5 min.
Pipette serum into labelled Eppendorf tube (ensuring not to disturb the blood clot).
Ensure Eppendorf tubes containing the serum are closed properly and place into plastic bag.
Store cold (4-8° C.) until ready for transportation. If freezing place in freezer as soon as possible.
Fill in details on Sample Submission Form and include with samples.
n=30 fish from each site are taken (n=10 fish from 3 cages).

### Sample Transportation Protocol

### Fresh Serum Samples:

### Materials:

Thermal postal pockets.
Freezer pack.
Postal bag

### Method:

Place freezer pack FLAT in the freezer and ensure frozen before use (if not placed flat will not fit into thermal postal pocket).
Place freezer pack at bottom of thermal postal pocket, followed by bag containing serum samples.
Seal thermal postal pocket and place into postal bag.
Post sample using next day delivery.

### Frozen Serum Samples:

### Materials:

Polystyrene box.
Freezer packs.
Ice

### Method:

Place freezer pack flat in the freezer and ensure frozen before use (if not placed flat will not fit into polystyrene box).
Place freezer pack(s) at bottom of polystyrene box, cover with layer of ice.
Place bag(s) of samples into ice.
Cover samples with ice and place freezer pack(s) on top. Ensure box is full.
Seal box and post using next day delivery.

### Sample Processing

Upon arrival in the lab the frozen samples are put into the −80° C. freezer for later batch analysis. The fresh samples are centrifuged (10 min at 1,200 g) to remove any suspended material, and the supernatant pipetted into the sample cup, observed and given a haemolysis score.

### Clinical Chemistry Measurement Including Optimisation for Fish Blood Serum

The software to operate the clinical chemistry instrument is ‘opened’ by the manufacturer to enable (where needed) to change the different settings for the clinical chemistry endpoint being investigated (see list of endpoints in Table 2). The specific endpoints that have been amended are listed below (Table 3). The reactive ranges of the fish samples are achieved by increasing or reducing the volume of serum sample added to the test. Other than this the tests are undertaken following the manufacturer's instructions, using all the relevant QC and calibration materials.

TABLE 3
The clinical chemistry assays amended to
fall within the reactive range of fish.

| Clinical chemistry assay | Old Vol (μl) | New Vol (μl) |
|---|---|---|
| Amylase (AMY) | 10 | 5 |
| Lactate dehydrogenase (LDH) | 3 | 2 |
| Phosphorous (P) | 5 | 2.5 |
| Potassium (K) | 5 | 10 |
| High Density Lipoprotein (LDL) | 3 | 2 |
| Creatine Kinase Myocardial band (CKMB) | 6 | 3 |

These endpoints have been measured on several different clinical chemistry instruments supplied by different manufacturers. These are:

### Randox Scientific:

### Instrument: RX Daytona Clinical Chemistry Analyser

TABLE 4
Randox Clinical chemistry assays.

| CAT. NO. | DESCRIPTION |
|---|---|
| AB3800 | Albumin (ALB) |
| AL3801 | Alanine Aminotransferase (ALT) |
| AM3979 | Ammonia (AMMON) |
| AP3802 | Alkaline Phosphatase (ALP) (AMP) (IFCC) |
| AP3803 | Alkaline Phosphatase (ALP) (DEA) (DGKC) |
| AY3805 | Amylase (AMY) |
| BR3859 | Total Bilirubin (TBIL) (JENDRASSIK) |
| BR4061 | Total Bilirubin (TBIL) (VANADATE OXIDATION) |
| CA3871 | Calcium (Ca2+) |
| CK3813 | Creatine Kinase (CK-MB) |
| CK3892 | Creatine Kinase (CK-NAC) |
| CK3878 | Creatine Kinase (CK-NAC) |
| CL1645 | Chloride (Cl−) |
| CR2336 | Creatinine (CREAT) |
| CU2340 | Copper (Cu) |
| GT3874 | γ -Glutamyltransferase (GGT) |
| GT3817 | γ -Glutamyltransferase (GGT) |
| HG1539 | Haemoglobin (MANUAL USE ONLY) |
| HA3450 | HAEMOGLOBIN DENATURANT REAGENT |
| LD3818 | Lactate Dehydrogenase (LDH) |
| MG3880 | Magnesium (Mg) |
| NA3851 | Sodium (Na+) |
| PH3820 | Phosphorus (PHOS) |
| PH3872 | Phosphorus (PHOS) |
| PT3852 | Potassium (K+) |
| SD125 | Ransod (Superoxide Dismutase) (SOD) |
| SI3821 | Iron (Fe) |
| TF3831 | Transferrin |
| TI4064 | Total Iron Binding Capacity (TIBC) |
| TP3869 | Total Protein (TP) |
| UR3825 | Urea (UR) |
| UR3873 | Urea (UR) |
| ZN2341 | Zinc |

### Fortress Diagnostics Ltd:

### Instrument: Monarch 400 & Monarch 240

TABLE 5
Fortress Diagnostics Clinical chemistry assays.

| Cat No. | Description |
|---|---|
| ALB (MON0222B) | Albumin |
| ALP (MON0185D) | Alkaline Phosphatase - Total |
| AMY (MON0563C) | Amylase |
| AST (MON0128A) | Aspartate Amino-Transferase |
| TBIL (MON0192A) | Bilirubin |
| Ca (MON0292B) | Calcium |
| K (MON0135A) | Potassium |
| Na (MON0142A) | Sodium |
| Total Cholesterol (MON0261H) | Cholesterol |
| CK (MON0252D) | Creatine Kinase - Total |
| CK-MB (MON0452D) | CK-MB |
| CO2 (MON0152C) | Carbon Dioxide |
| CRE (MON0117A) | Creatinine Jaffe |
| Glucose (MON0101H) | Glucose |
| HDL (MON0421A) | HDL-Cholesterol |
| Fe Ferozine (MON0235C) | Iron |
| LACT (MON0622A) | Lactate |
| LDH (MON0242C) | Lactate Dehydrogenase |
| LDL (MON0431G) | LDL-Cholesterol |
| Lipase | Lipase |
| Mg (MON0352A) | Magnesium |
| AMM (MON0376A) | Ammonia |
| P (MON0302A) | Phosphate inorganic/Phosphorus |
| TP (MON0173B) | Total Protein |
| Trigycerides (MON0271A) | Triglycerides |
| Cl (MON0281A) | Chloride |
| Zn (MON0462B) | Zinc |
| ALT (MON0127A) | Alanine aminotransferase |
| Cu (MON0341A) | Copper |
| TIBC (MON0237B) | Total iron binding capacity |
| ALDO (MON0391A) | Aldolase |
| Bile Acids (MON0584A) | Bile acids |
| Cholinesterase (MON0801C) | Cholinesterase |
| Uric Acid (MON0602C) | Uric acid |

### Roche Diagnostics Ltd.:

Instrument: Cobas C 311 analyser

TABLE 6
Roche Clinical chemistry assays.

| Cat No. | Description |
|---|---|
| ALB2, 750T | Albumin |
| ALP2, 1100T | Alkaline Phosphatase - Total |
| AMYL2, 750T | alpha-Amylase |
| ASTP2, 800T | Aspartate Amino-Transferase |
| BILT3, 1050T | Bilirubin |
| CA2, 1500T | Calcium |
| Cartridge K | Potassium |
| Cartridge NA | Sodium |
| CHOL2, 2600T | Cholesterol |
| CK, 500T | Creatine Kinase - Total |
| CKMB, 150T | CK-MB |
| CO2-L, 250T | Carbon Dioxide |
| CREAJ2, 2500T | Creatinine Jaffe |
| CRP4, 500T | C-Reactive Protein |
| GLUC3, 3300T | Glucose |
| HDLC4, 700T | HDL-Cholesterol |
| IRON2, 700T | Iron |
| LACT2, 100T | Lactate |
| LDHI2, 850T | Lactate Dehydrogenase |
| LDLC3, 600T | LDL-Cholesterol |
| LIPC, 200T | Lipase |
| MG2, 690T | Magnesium |
| NH3L2, 300T | Ammonia |
| PHOS2, 750T | Phosphate inorganic/Phosphorus |
| TP2, 1050T | Total Protein |
| TRIGL, | Triglycerides |

### Data Clinical Interpretation

Data generated from the clinical chemistry analyser is extracted and added to a database. As there is currently no data on the normal ranges found throughout the year for the present biomarkers in salmonids and therefore no current method to clinically interpret the data. The present invention have established normal ranges of each biomarker for aquaculture reared salmonids. As with normal practice in human medicine, the mean±1 SD is used to establish the normal range (green in our traffic light system), with ±1 to 2 SDs representing our abnormal range (amber) and >2 SDs representing unhealthy samples (red) (see FIG. 7). These calculations were undertaking using the Reference Value Advisor software. These background levels are used for the clinical interpretation of the results generated for the fish serum.

Where possible, samples are also compared against healthy ‘control’ samples taken from the same site. An example of this is where a health challenge has been positively identified (primarily using PCR or histopathology) as having a health challenge, whereas another pen from the site does not (control pen). Comparison of the data generated from both pens offers an opportunity to directly measure the impact of the health challenge on the clinical chemistry expression in the fish.

For both continuous health monitoring and diagnostic testing results are presented within 24 h of sample arrival to Laboratory via website login portal. Results are presented to include:
I. Fish health assessment based on bespoke AI model (healthy v unhealthy) and health challenge identification
II. Specific health challenge assessment
III. Graphs of individual biomarkers compared against our bespoke biomarker background levels and where possible, against non-infected ‘control’ samples
IV. Raw data presented via excel spreadsheet

### Example 1: Data Clinical Interpretation Results

### Pancreas Disease (PD)

Samples were collected at four sampling points, with heathy fish (from 4 cages, n=10 per cage) compared against PD positive and PD recovered fish (8 cages, n=10 per cage).

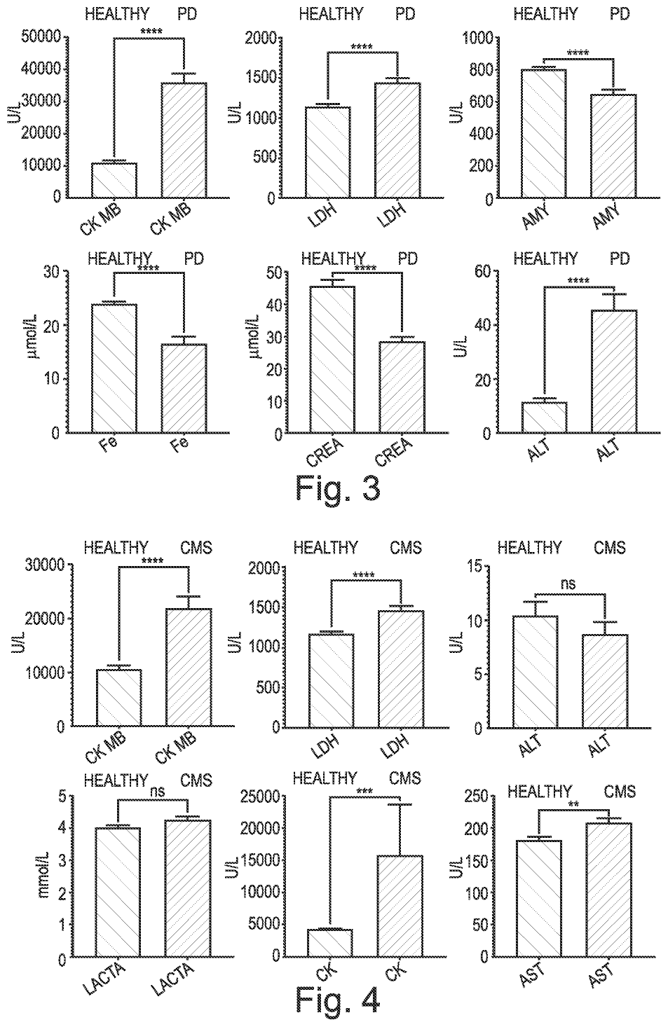

Biomarkers that showed an increased expression in PD fish included creatine kinase-MB (CK MB) and Lactate dehydrogenase (LDH) and alanine aminotransferase (ALT). Biomarkers with decreased expression in PD infected fish include Amylase (Amy), Creatinine (Crea) and Iron (Fe) (see FIG. 3).

PD recovered fish as used herein may refer to fish that have returned to feeding but still display characteristics of pancreas disease. Suitably, in some embodiments PD recovered fish have increased expression of creatine kinase-MB (CK MB), Lactate dehydrogenase (LDH) and alanine aminotransferase (ALT) and decreased expression in Amylase (Amy), Creatinine (Crea) and Iron (Fe). Suitably, in some embodiments the changes in expression e.g. increased expression of creatine kinase-MB (CK MB), Lactate dehydrogenase (LDH) and alanine aminotransferase (ALT) and decreased expression in Amylase (Amy), Creatinine (Crea) and Iron (Fe) are not to the same extent as PD infected fish, with expression in the amber range (as opposed to the red range).

### Cardiomyopathy Syndrome (CMS)

Samples were collected from two sampling points with healthy fish (4 cages, n=10 per cage) compared against CMS positive fish (8 cages, n=10 per cage). Biomarkers that showed an increased expression in CMS infected fish include Creatine kinase-MB (CK MB), Lactate dehydrogenase (LDH), Lactate (LACTA), Creatine kinase (CK) and Aspartate aminotransferase (AST). Biomarkers with decreased expression in CMS infected fish include Alanine aminotransferase (ALT) (see FIG. 4).

### Gill Issues

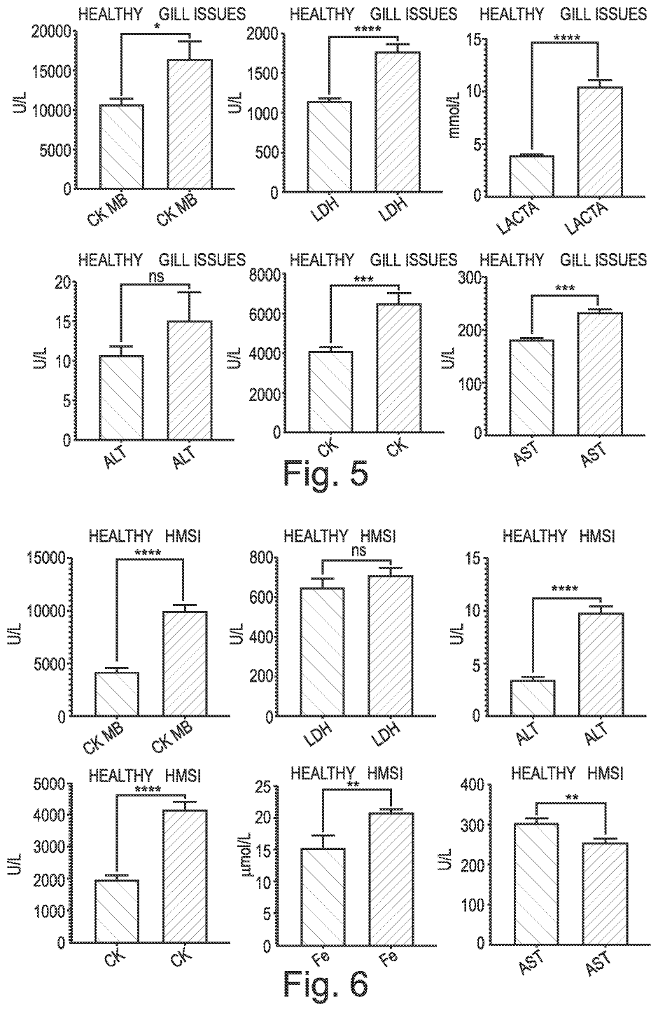

Samples were collected from one sampling point with healthy fish (4 cages, n=10 per cage) compared against fish with confirmed gill issues (4 cages, n=10 per cage). Biomarkers that showed an increased expression in fish with compromised gills include Creatine kinase-MB (CK MB), Lactate dehydrogenase (LDH), Lactate (LACTA), Alanine aminotransferase (ALT), Creatine kinase (CK), and Aspartate aminotransferase (AST) (see FIG. 5).

### Heart and Skeletal Muscle Inflammation (HMSI)

Samples were collected from 4 sampling points with healthy fish (3 cages, n=10 per cage) compared against HSMI confirmed fish (3 cages, n=10 per cage).

Biomarkers that showed an increased expression in HSMI confirmed fish include Creatine kinase-MB (CK-MB), Lactate dehydrogenase (LDH), Alanine aminotransferase (ALT), Creatine kinase (CK), and Iron (Fe). Biomarkers with decreased expression in CMS infected fish include Aspartate aminotransferase (AST) (see FIG. 6).

### Example 2: Biomarker Background Levels for Clinical Interpretation

The background levels from salmonids (n=1,525) for several of the biomarkers investigated were calculated. FIG. 7 demonstrates the background levels in aquaculture reared salmonids for lactate dehydrogenase, creatine kinase, creatine kinase-MB, alanine aminotransferase, aspartate aminotransferase and phosphorous respectively. Grey represents the healthy range the mean±1 SD, with ±1 to 2 SDs representing our abnormal range (light grey) and >2 SDs representing unhealthy samples (dark grey).

### Example 3: Data Interpretation/Classification Using AI Modelling

Introduction: The aim was to develop an AI framework using machine learning techniques to predict some common fish diseases of an individual fish from their blood biochemistry parameters (full list in Table 2). The idea is to create predictive models that are capable of monitoring disease progression from the change in blood biochemistry markers.

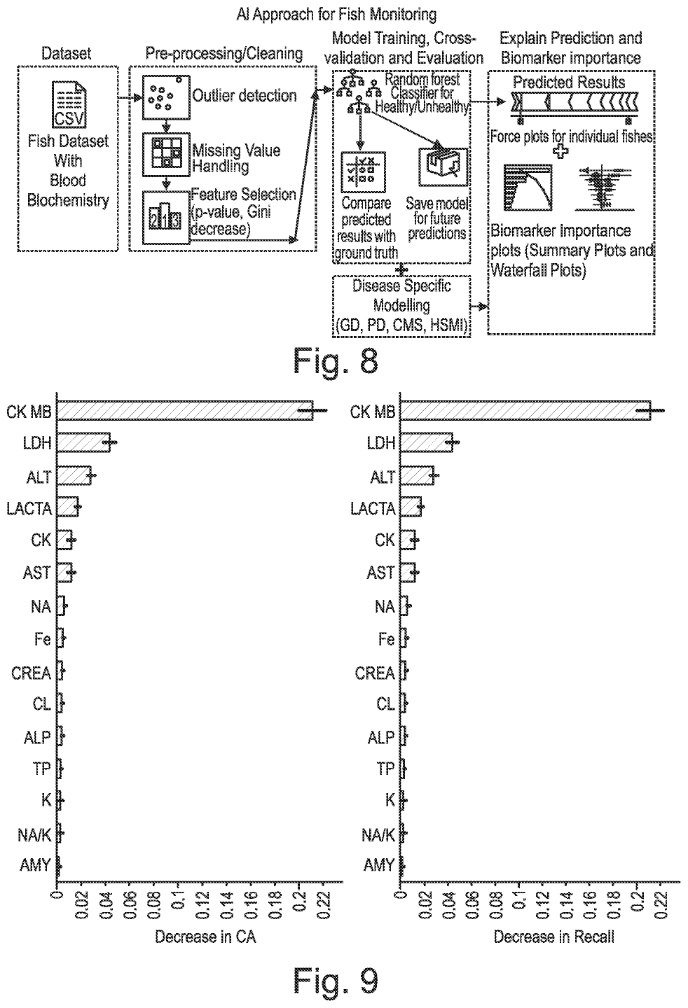

Method: Visualization interfaces and Machine Learning models/tools/algorithms were developed to identify the effect of various biomarkers on fish health. To a dataset of fish health biochemistry, pre-processing techniques, outlier detection, missing value handling and feature selection methods were applied. An algorithm was then trained to distinguish between healthy and unhealthy, using standard validation and evaluation techniques before the algorithm was saved and used for predicting the labels of the test data. A schematic diagram of the AI modelling approach for fish health monitoring is provided in FIG. 8.

### Outlier Detection:

Data values>1.5 IQR (interquartile range) is selectively dropped so that the imputation station stage does not get biased by these values. A Local outlier detection algorithm can be used on more data.

### Missing Value Imputation:

The missing values are filled with class-wise average/median values for the biomarkers. Advance model based multiple imputation techniques can also be used.

Feature Selection (p-Value, Gini Decrease):

Feature selection started with 25 biomarkers and the number of biomarkers were gradually reduced and their effect monitored on model accuracy and finally settled at 15 biomarkers (using Gini-decrease) chosen from a pool of 20 biomarkers, after assessing the trade-off between computational accuracy and prediction time. Processing fewer biomarkers provides faster computation time.

### Random Forest Algorithm Classifier for Healthy/Unhealthy:

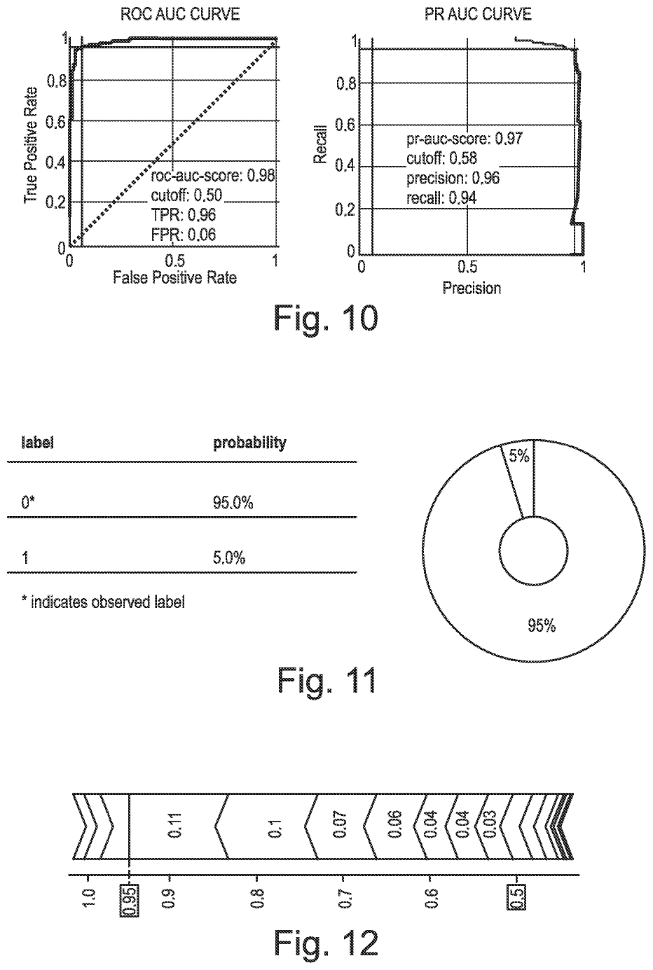

A tailored random forest classifier with stacking sub-models was trained to ensure accurate prediction above 95% for both Healthy and Unhealthy classes. FIGS. 9 and 10 show the effect of the 15 biomarkers on the models performance.

The can produce class labels with associated class-wise probability/confidence value for its decision (see FIG. 11).

### Fish Health Measuring Scale Using Force Plots:

The inventors have deployed an explainable AI library called SHAP to produce further breakdown on how each feature has contributed to the prediction (FIG. 12).

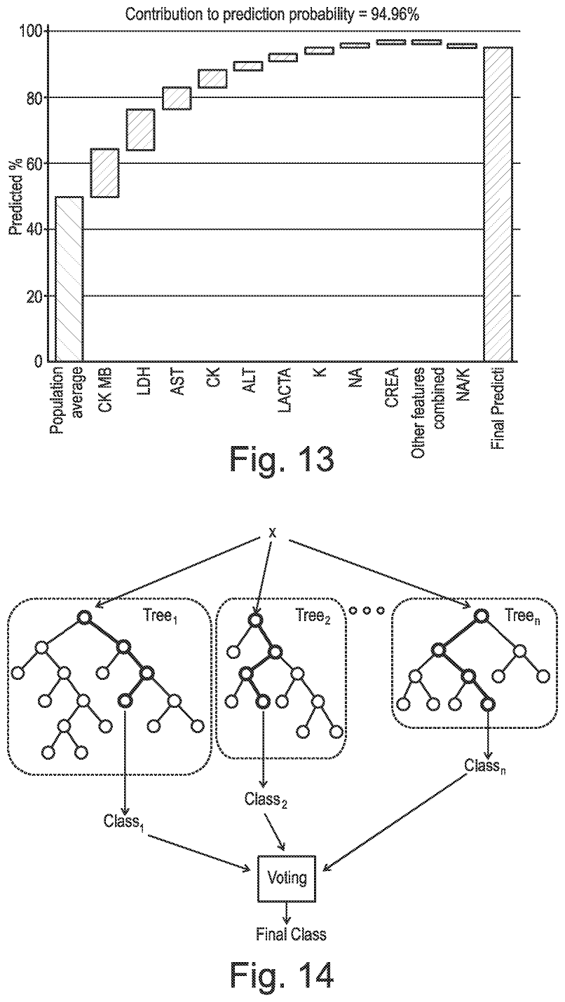

For the prediction shown above where the model predicts with 95% probability that the fish is healthy, the contribution from the biomarkers. Using biomarker contribution plot, the model can measure fish health (see FIG. 13).

### Example 4: Disease Specific Multi-Class Modelling

The model currently has disease specific sub-models boosting the performance of the basic random forest. A stacked multi-class disease model was deployed using the list of 15 selected biomarkers where sub models are created using filtered portion of unhealthy fish samples with individual diseases (see Table 7). The diseases covered by the model are, cardiomyopathy syndrome (CMS), complex gill disease (CGD)/gill issues, osmoregulation issues, heart and skeletal muscle inflammation (HSMI), pancreas disease (PD) and other health issues combined under an Unhealthy Other class at this point of time. The disease class labels are generated by domain experts from the associated fish-farms (based on PCR and histopathology assessment) and a clinical biochemistry expert.

TABLE 7
Samples available for various disease/unhealthy groups

| Model-1 Classes | HEALTHY | UNHEALTHY | Total |
|---|---|---|---|
| CMS | 0 | 34 | 34 |
| GILL ISSUES | 0 | 173 | 173 |
| HEALTHY | 681 | 0 | 681 |
| HSMI | 0 | 74 | 74 |
| OSMOREGULATION | 0 | 31 | 31 |
| ISSUES | | | |
| PD | 0 | 75 | 75 |
| UNHEALTHY OTHER | 0 | 76 | 76 |
| Total | 681 | 463 | 1144 |

### Base-Classifiers—Random Forest Algorithm:

For model implementation the present invention uses popular ensemble classifier called the Random Forest (Breiman, 2001) which operates by constructing multiple decision tree models at the training time. It is one of the most accurate supervised learning methods in recent times. Each decision tree in a Random Forest represents one class of observations that are being considered. Decision trees are constructed during the learning process with the training data.

Random Forests mainly rely upon two parameters to control their growth: numTrees, the number of decision trees to be built and numFeatures, the number of random subset of features to assess at each tree node (Devetyarov, et al., 2010).

In the present design, numTrees=10 and numFeatures=15. Each of the 10 decision trees is constructed in a top-down manner starting with a root node by selecting a set of N observations of size n at random with replacement from the training dataset and selecting the most significant features of these samples as the tree nodes. At each node a, the m number of features is selected at random from 15 features to grow the tree and the most significant feature that provides the best binary split on that node is selected among all according to an objective function. Feature significance is generally estimated using the Gini index (Ogwant, T., 2014). To classify a new sample, the features/biomarker values of the samples are tested with each of the decision trees present in the random forest. Each tree gives a classification score or “vote” and the class with the most votes is selected as the class to which the sample belongs. The voting process is illustrated in FIG. 14. The method used the RandomForestClassifier from the sklearn.ensemble module in python for training the models (Feurer, M. et al., 2018).

### Stacking Classifiers for Higher Predictive Performance:

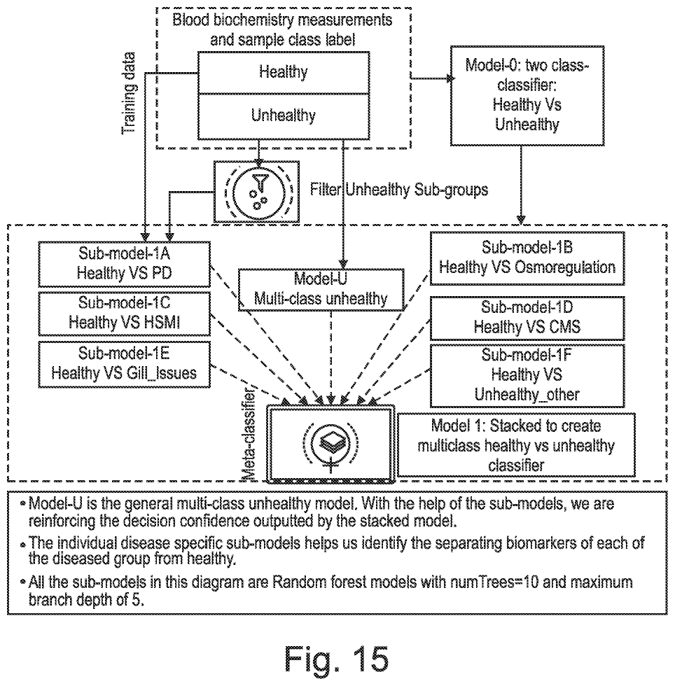

The simplest form of stacking can be described as an ensemble learning technique where the predictions of multiple classifiers are used as new features to train a meta-classifier (Feurer, M. et al., 2018). FIG. 15 is demonstrating the stacking scheme used to train and implement our multi-class disease models. The meta-classifier of our choice is a logistic regression model. In FIG. 15, Model-U is the general multi-class unhealthy model that is capable of distinguishing one disease from the other and is trained on ‘Unhealthy’ portion of the training data only. Once this model is built, it was noticed some unhealthy groups such as the ones with cardiomyopathy syndrome (CMS), complex gill disease (CGD)/gill health demonstrate quite a high amount of confusion in their decision making (see Table 8). Moreover, the biomarker differences from healthy samples to specific unhealthy groups are greater than one unhealthy group to another. Hence to increase the model confidence, the inventors constructed multiple healthy vs disease sub-models to distinguish each unhealthy group from the reference healthy class. The individual disease specific sub-models (FIG. 15) helped to identify the separating biomarkers of each of the diseased group from healthy (see Tables 8-14). By stacking all these sub-models, the inventors managed to reinforce and the decision confidence of the final stacked model in this way. All the sub-models in this diagram are Random forest models with numTrees=10 and has maximum branch depth of 5 in the individual decision trees to keep the computation fast enough during prediction stage. For the implementation the StackingClassifier from the mlxtend.classifier module were used (Hatami, N & Ebrahimpour, R., 2007).

TABLE 8
Model-U Confusion Matrix

| | | CGD/GILL | | Osmoregulation | | Unhealthy | |
|---|---|---|---|---|---|---|---|
| Actual/predicted | CMS | ISSUES | HSMI | issues | PD | other | Σ |
| CMS | 70.00% | 6.60% | 0.00% | 3.70% | 7.00% | 0.00% | 34 |
| Gill health | 20.00% | 75.30% | 5.50% | 0.00% | 7.00% | 16.90% | 173 |
| HSMI | 0.00% | 4.00% | 89.00% | 3.70% | 0.00% | 0.00% | 74 |
| Osmoregulation | 0.00% | 1.00% | 4.10% | 92.60% | 1.20% | 0.00% | 31 |
| issues | | | | | | | |
| PD | 10.00% | 2.50% | 0.00% | 0.00% | 75.60% | 5.10% | 75 |
| Unhealthy | 0.00% | 10.60% | 1.40% | 0.00% | 9.30% | 78.00% | 76 |
| other | | | | | | | |
| Σ | 20 | 198 | 73 | 27 | 86 | 59 | 463 |

TABLE 9
Sub-model-1A HEALTHY vs PD Confusion Matrix

| | predicted | | |
|---|---|---|---|
| Actual | HEALTHY | PD | Σ |
| HEALTHY | 99.40% | 0.00% | 681 |
| PD | 0.60% | 100.00% | 75 |
| Σ | 685 | 71 | 756 |

TABLE 10
Sub-model-1B HEALTHY vs OSMOREGULATION
ISSUES Confusion Matrix

predicted

| | | Osmoregulation | |
|---|---|---|---|
| Actual | Healthy | issues | Σ |
| Healthy | 99.70% | 3.30% | 681 |
| Osmoregulation issues | 0.30% | 96.70% | 31 |
| Σ | 682 | 30 | 712 |

TABLE 11
Sub-model-1C HEALTHY vs HSMI Confusion Matrix

| | predicted | | |
|---|---|---|---|
| Actual | Healthy | HSMI | Σ |
| Healthy | 100.00% | 0.00% | 681 |
| HSMI | 13.50% | 86.50% | 74 |
| Σ | 691 | 64 | 755 |

TABLE 12
Sub-model-1D HEALTHY vs CMS Confusion Matrix

| | predicted | | |
|---|---|---|---|
| Actual | CMS | Healthy | Σ |
| CMS | 70.60% | 29.40% | 34 |
| Healthy | 0.10% | 99.90% | 681 |
| Σ | 25 | 690 | 715 |

TABLE 13
Sub-model-1E HEALTHY vs CGD/GILL
ISSUES Confusion Matrix

predicted

| Actual | CGD/GILL ISSUES | Healthy | Σ |
|---|---|---|---|
| CGD/GILL ISSUES | 89.00% | 11.00% | 173 |
| Healthy | 1.20% | 98.80% | 681 |
| Σ | 162 | 692 | 854 |

TABLE 14
Sub-mode-1F HEALTH vs UNHEALTHY
OTHER Confusion Matrix

| | predicted | | |
|---|---|---|---|
| Actual | Healthy | Unhealthy other | Σ |
| Healthy | 99.90% | 0.10% | 681 |
| Unhealthy other | 21.10% | 78.90% | 76 |
| Σ | 696 | 61 | 757 |

TABLE 15
Model-1 overall Confusion Matrix, operating on the entire dataset, in five-fold cross-validation

| | | CGD/Gill | | | Osmoregulation | | Unhealthy | |
|---|---|---|---|---|---|---|---|---|
| Actual/predicted | CMS | issues | Healthy | HSMI | Issues | PD | other | Σ |
| CMS | 71.60% | 3.90% | 0.40% | 0.00% | 3.40% | 7.60% | 0.00% | 34 |
| CGD/Gill ISSUES | 21.40% | 78.50% | 1.40% | 3.00% | 0.00% | 3.80% | 14.70% | 173 |
| HEALTHY | 0.00% | 5.00% | 96.10% | 1.50% | 0.00% | 0.00% | 2.90% | 681 |
| HSMI | 0.00% | 2.20% | 1.10% | 92.40% | 3.40% | 0.00% | 0.00% | 74 |
| OSMOREGULATION | 0.00% | 0.60% | 0.00% | 3.00% | 93.10% | 1.30% | 0.00% | 31 |
| ISSUES | | | | | | | | |
| PD | 8.00% | 2.20% | 0.10% | 0.00% | 0.00% | 77.20% | 10.30% | 75 |
| UNHEALTHY | 0.00% | 7.70% | 0.70% | 0.00% | 0.00% | 10.10% | 72.10% | 76 |
| OTHER | | | | | | | | |
| Σ | 25 | 181 | 696 | 66 | 29 | 79 | 68 | 1144 |

### Data Interpretation/Classification Using AI Modelling Results

The specific biomarkers used in model 0 and model 1 for each health challenge in order of importance for each model are shown in table 15.

TABLE 16
Top biomarkers for determining healthy v unhealthy
fish and identifying specific health challenges.

| | Top-20 Important | | | | | |
|---|---|---|---|---|---|---|
| | Biomarkers (Gini | | | | | |
| | Index) | 15 Biomarkers/health challenge | | | | |
| | Healthy V | | | | GILL | OSMOREGULATION |
| | Unhealthy | PD | CMS | HSMI | HEALTH | ISSUES |
| Order | MODEL 0 | MODEL 1 | MODEL 1 | MODEL 1 | MODEL 1 | MODEL1 |
| 1 | LDH | CK MB | CK MB | CK MB | CK MB | CK MB |
| 2 | CK | LDH | LDH | LDH | LDH | CK |
| 3 | CK MB | AMY | ALT | ALT | LACTA | TP |
| 4 | ALT | Fe | LACTA | CK | ALT | Fe |
| 5 | AST | CREA | CK | Fe | CK | LDH |
| 6 | K | ALT | AST | AST | AST | AMY |
| 7 | NA/K | CK | ALP | LACTA | Fe | ALT |
| 8 | LACTA | LACTA | NA | K | CREA | AST |
| 9 | AMY | TP | Fe | AMY | ALP | CL |
| 10 | CREA | K | K | CL | NA | Na/K |
| 11 | TP | Na/K | CREA | ALP | AMY | Na |
| 12 | P | ALP | TP | Na | TP | LACTA |
| 13 | NA | Na | AMY | TP | K | K |
| 14 | ZN | AST | CL | CREA | Na/K | CREA |
| 15 | AMM | CL | NA/K | Na/K | CL | ALP |

Creatine kinase (CK); Creatine kinase-MB (CK-MB); Lactate dehydrogenase (LDH); Aspartate aminotransferase (AST); Alanine aminotransferase (ALT); Lactate (LACTA); Total Protein (TP); Alkaline Phosphatase (ALP); Creatinine (CREA); Amylase (AMY); Phosphorus (P); Iron (Fe); Zinc (Zn); Potassium (K); Sodium (Na): Sodium/Potassium (Na/K); Chloride (Cl); Ammonia (AMM).

### Discussion

Through the repurposing of medical clinical chemistry instrumentation and assays the establish background levels for 33 biomarkers in aquaculture reared salmonid fish (Atlantic salmon (Salmo salar) and rainbow trout (Oncorhynchus mykiss) have been established. This information was then used to clinically interpret results to establish the impacts of various health challenges. This data was also used to create an AI model to categorise the fish as health or unhealthy and to further classify the specific health challenge based on the expression of a suite of biomarker. This approach is largely automated enabling the results presentation back to the fish farmer within 24 h of sample receipt to the lab. This unique approach to fish health is both non-lethal and fast which provides a significant advantage over the slow and lethal histopathology-based methods that the aquaculture industry is currently reliant upon.

### REFERENCES

1. Adel, M., Abedian Amiri, A., Zorriehzahra, J., Nematolahi, A. & Esteban, M. Á. 2015. Effects of dietary peppermint (Mentha piperita) on growth performance, chemical body composition and hematological and immune parameters of fry Caspian white fish (Rutilus frisii kutum). Fish Shellfish Immunol. 45 (2), 841-7.
2. Barisic, J, Cannon, S., Quinn, B. 2019. Cumulative impact of anti-sea lice treatment (azamethiphos) on health status of Rainbow trout (Oncorhynchus mykiss, Walbaum 1792) in aquaculture. Scientific Reports volume 9, Article number: 16217.
3. Benfey, T. J. & Biron, M. 2000. Acute stress response in triploid rainbow trout (Oncorhynchus mykiss) and brook trout (Salvelinus fontinalis). Aquaculture 184, 167-176.
4. Bernet, D., Schmidt-Posthaus, H., Wahli, T. & Burkhardt-Holm, P. 2000. Effects of wastewater on fish health: an integrated approach to biomarker responses in brown trout (Salmo trutta L.). J. Aquat. Ecosyst. Stress Recover. 8, 143-151.
5. Braceland, M., Houston, K., Ashby, A., Matthews, C., Haining, H., Rodger, H., Eckersall, P. D. 2017. Technical pre-analytical effects on the clinical biochemistry of Atlantic salmon (Salmo salar L.). Journal of Fish Diseases 2017, 40, 29-40.
6. FAO 2020: The State of World Fisheries and Aquaculture 2020 report by the United Nations Food and Agriculture Organisation (UN FAO).
7. Breiman, Leo. Random forests. Machine learning 45.1 (2001): 5-32.
8. Devetyarov, D., and Nouretdinov, I. 2010. Prediction with confidence based on a random forest classifier. IFIP International Conference on Artificial Intelligence Applications and Innovations. Springer, Berlin, Heidelberg.
9. Ferri, J. et al. 2011. The effect of artificial feed on blood biochemistry profile and liver histology of wild saddled bream, Oblada melanura (Sparidae). Mar. Environ. Res. 71, 218-224.
10. Floyd-Rump, T. P., Horstmann-Dehn, L. A., Atkinson, S. & Skaugstad, C. 2017. Effect of ichthyophonus on blood plasma chemistry of spawning chinook salmon and their resulting offspring in a yukon river tributary. Dis. Aquat. Organ. 122 (3), 223-236.
11. Feurer, Matthias, et al. Auto-sklearn (2018): efficient and robust automated machine learning. Automated Machine Learning: 113-134.
12. Hatami, Nima, and Reza Ebrahimpour. 2007. Combining multiple classifiers: diversify with boosting and combining by stacking. International Journal of Computer Science and Network Security 7.1:127-131.
13. Hille, S. 1982. A literature review of the blood chemistry of rainbow trout, Salmo gairdneri Rich. J. Fish Biol. 20, 535-569.
14. Javed, M., Ahmad, M. I., Usmani, N. & Ahmad, M. 2017. Multiple biomarker responses (serum biochemistry, oxidative stress, genotoxicity and histopathology) in Channa punctatus exposed to heavy metal loaded wastewater. Sci. Rep. 7, 1765.
15. Munro L. A. & Wallace, I. S., 2017. Scottish fish farm production seruvery 2017. Scottish Government, 2018.
16. Ogwang, Tomson. A convenient method of decomposing the Gini index by population subgroups. Journal of Official Statistics 30.1 (2014): 91.
17. Quinn, B., McEneff, G., Schmidt, W. 2015. Pharmaceuticals in the Irish Aquatic Environment: The Assessment and Potential Human Impact of Exposure to Pharmaceuticals on Marine and Freshwater Bivalves. EPA (Ireland) report 143. 40 pp.
18. Řehulka, J. 2003. Haematological analyses in rainbow trout Oncorhynchus mykiss affected by viral haemorrhagic septicaemia (VHS). Dis. Aquat. Organ. 56, 185-193.
19. Sandnes K. L. & Waagbo, R. 1988. Normal ranges of some blood chemistry parameters in adult farmed Atlantic salmon, Salmo salar. Journal of Fish Biology (1988) 32 (1) 129-136.
20. Steinbach, C. et al. 2014. The sub-lethal effects and tissue concentration of the human pharmaceutical atenolol in rainbow trout (Oncorhynchus mykiss). Sci. Total Environ. 497-498, 209-218.

1. A method for determining health status of a population of fish, comprising the steps of:
(a) analysing a first sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the sample to determine a test profile; and
(b) comparing the amount of the at least one analyte present in the test profile with a reference profile;
wherein a difference in the amount of the at least one analyte in the test profile as compared to the reference profile indicates the health status of the population of fish.

2. A method for monitoring health status of a population of fish, comprising the steps of:
(a) analysing a first sample collected from at least one fish of the population of fish at a first time point to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile; and
(b) analysing at least a second sample collected from at least one fish of the population of fish to determine the amount of the same at least one analyte present in the at least second sample to determine a second test profile;
(c) comparing the amount of the second test profile with a reference profile;
wherein a difference in the amount of the at least one analyte between the first and/or second test profile with the reference profile indicates a change in the health status of the population of fish.

3. The method according to claim 1, wherein the method comprises analysing a sample collected from said population of fish to determine the amount of lactate dehydrogenase and from none to one or more further analytes selected from the group consisting of: creatine kinase; creatine kinase-MB; alanine aminotransferase; aspartate aminotransferase; potassium; sodium/potassium ratio; lactate; amylase; creatinine; total protein; phosphorous; sodium; zinc; and ammonia.

4. The method according to claim 1, wherein the method comprises analysing a sample collected from said population of fish to determine the amount of lactate dehydrogenase and creatine kinase as well as anywhere from none to one or more further analytes selected from the group consisting of: creatine kinase-MB; alanine aminotransferase; aspartate aminotransferase; potassium; sodium/potassium ratio; lactate; amylase; creatinine; total protein; phosphorous; sodium; zinc; and ammonia.

5. The method according to claim 1, wherein the method comprises analysing a sample collected from said population of fish to determine the amount of lactate dehydrogenase, creatine kinase and creatine kinase-MB as well as anywhere from none to one or more further analytes selected from the group consisting of: alanine aminotransferase; aspartate aminotransferase; potassium; sodium/potassium ratio; lactate; amylase; creatinine; total protein; phosphorous; sodium; zinc; and ammonia.

6. The method according to claim 1, wherein the method comprises analysing a sample collected from said population of fish to determine the amount of lactate dehydrogenase, creatine kinase, creatine kinase-MB, and alanine aminotransferase as well as anywhere from none to one or more further analytes selected from the group consisting of: aspartate aminotransferase; potassium; sodium/potassium ratio; lactate; amylase; creatinine; total protein; phosphorous; sodium; zinc; and ammonia.

7. The method according to claim 1, wherein the method comprises analysing a sample collected from said population of fish to determine the amount of lactate dehydrogenase, creatine kinase, creatine kinase-MB, alanine aminotransferase, and aspartate aminotransferase as well as anywhere from none to one or more further analytes selected from the group consisting of: potassium; sodium/potassium ratio; lactate; amylase; creatinine; total protein; phosphorous; sodium; zinc; and ammonia.

8. The method according to claim 1, wherein the method comprises analysing a sample collected from said population of fish to determine the amount of lactate dehydrogenase, creatine kinase, creatine kinase-MB, alanine aminotransferase, and aspartate aminotransferase as well as anywhere from none to one or more further analytes selected from the group consisting of: potassium; sodium/potassium ratio; lactate; amylase; creatinine; total protein; phosphorous; sodium; zinc; and ammonia.

9. The method according to claim 1, wherein the method comprises analysing a sample collected from said population of fish to determine the amount of lactate dehydrogenase, creatine kinase, creatine kinase-MB, alanine aminotransferase, aspartate aminotransferase and potassium as well as anywhere from none to one or more further analytes selected from the group consisting of: sodium/potassium ratio; lactate; amylase; creatinine; total protein; phosphorous; sodium; zinc; and ammonia.

10. The method according to claim 1, wherein the method comprises analysing a sample collected from said population of fish to determine the amount of lactate dehydrogenase, creatine kinase, creatine kinase-MB, alanine aminotransferase, aspartate aminotransferase and potassium.

11. The method of claim 1, wherein the sample is collected from at least one, at least two, at least three, at least four, at least five, at least six, at least seven, at least eight, at least nine, at least ten, at least twenty, at least thirty, at least forty, at least fifty or at least 100 fish from the population of fish.

12. The method of claim 11, wherein the sample is collected from a plurality of fish from the population of fish.

13. The method according to claim 1, wherein the method is performed prior to observing physical or behavioural characteristics of a condition or disease.

14. A method of diagnosing a population of fish with a condition or a disease, the method comprising:
(a) analysing a sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the sample to determine a test profile; and
(b) comparing the amount of the at least one analyte present in the test profile with a reference profile;
wherein a difference in the amount of the at least one analyte in the test profile as compared to the reference profile indicates the population of fish have a condition or a disease.

15. The method of claim 14, wherein an increase in creatine kinase-MB, lactate dehydrogenase and alanine aminotransferase, and a decrease in amylase, creatinine and iron indicates the population of fish have pancreas disease.

16. The method of claim 14, wherein an increase in creatine kinase-MB, lactate dehydrogenase, lactate, creatine kinase and aspartate aminotransferase, and a decrease in alanine aminotransferase indicates the population of fish have cardiomyopathy syndrome.

17. The method of claim 14, wherein an increase in creatine kinase-MB, lactate, lactate dehydrogenase, alanine aminotransferase, creatine kinase and aspartate aminotransferase indicates the population of fish have compromised gills.

18. The method of claim 14, wherein an increase in creatine kinase-MB, creatine kinase, lactate dehydrogenase, alanine aminotransferase and iron, and a decrease in aspartate aminotransferase indicates the population of fish have heart and skeletal muscle inflammation.

19. The method according to claim 14, wherein the condition or disease is any one or more of: dehydration, GI loss, renal disease, shock, circulatory failure, low blood sodium, metabolic alkalosis, metabolic acidosis, chronic kidney disease, pancreatitis, renal insufficiency, malabsorption, poor diet, loss of blood, anaemia, hepatitis, cirrhosis, haemolytic diseases, obstruction of biliary, hepatic and/or pancreatic ducts, impaired kidney function, kidney disease, liver disease, gill pathology, infections, protein loss, malnutrition, malignancy, starvation, infection, immunosuppression, haemolytic anaemia, inflammation, hepatitis, drug induced liver damage, heart damage, trauma, bone disease and periods of bone growth, hypothyroidism, pernicious anaemia, muscle trauma, skeletal and cardiac muscle damage, and haemorrhage.

20. A method for monitoring progression of a condition or disease in a population of fish, comprising the steps of:
(a) analysing a first sample collected from at least one fish of the population of fish at a first time point to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile; and
(b) analysing at least one later sample collected from at least one fish at least one later time point to determine the amount of the same at least one analyte present in the least one later sample to determine at least one later test profile;
(c) comparing the amount of the at least one analyte present in the at least one later test profile with a reference profile;
wherein a difference in the amount of the at least one analyte between the at least one later test profile with the reference profile indicates progression in the condition or disease.

21. The method according to claim 20, wherein progression in the condition or disease may be an improvement or a worsening in the condition or disease.

22. A method for determining whether or not to treat a population of fish, or determining whether a proposed treatment is appropriate, comprising the steps of:
(a) analysing a sample collected from at least one fish of the population of fish to determine the amount of at least one analyte selected from the group consisting of: lactate dehydrogenase; creatine kinase; creatine kinase-MB; alanine aminotransferase; aspartate aminotransferase; potassium; sodium/potassium ratio; lactate; amylase; creatinine; total protein; phosphorous; sodium; zinc; ammonia; alkaline phosphatase; iron; chloride; carbon dioxide; albumin; calcium; magnesium; total bilirubin; globulins; total iron binding capacity; copper; and total antioxidative status present in the first sample to determine a test profile;
(b) comparing the amount of the at least one analyte present in the test profile with a reference value;
wherein a difference in the amount of the at least one analyte between the test profile and the reference profile indicates the population of fish have developed a condition or a disease and should not be treated or an alternative treatment method recommended.

23. The method according to claim 22, wherein the population of fish should not be treated for a parasite infection or an alternative treatment method recommended.

24. The method of claim 23, wherein the population of fish should not be treated for a sea lice infection or an alternative treatment method recommended.

25. The method according to claim 1, wherein the population of fish have a change in health status or the method according to any one of claims 14-24 wherein the fish have a condition or disease, the method further comprises harvesting a population of fish.

26. A method according to claim 1, wherein the method further comprises providing an effective amount of a therapeutic agent for administration to the population of fish to treat the change in health status or the condition or disease.

27. (canceled)

28. The method according to claim 1, wherein the sample is blood, plasma or serum.

29. The method according to claim 1, wherein the fish or population of fish are wild, captive or farmed fish.

30. The method according to claim 29, wherein the fish or population of fish are salmonid, sea bass, sea bream, sturgeon and/or carp.

31. The method according to claim 1, wherein the amount of the at least one analyte being present in the abnormal and/or unhealthy analyte reference range indicates the population of fish have a change in health status and/or a condition or disease.

32. A kit for use in a method of claim 1, wherein the kit comprises one or more reagents for determining the amount of the at least one analyte in a sample, and instructions for use.

33. The method of claim 2, wherein the reference profile is the first test profile.

34. The method of claim 20, wherein the reference profile is the first test profile.

35. The method according to claim 2, wherein the method comprises analysing a sample collected from said population of fish to determine the amount of lactate dehydrogenase and from none to one or more further analytes selected from the group consisting of: creatine kinase; creatine kinase-MB; alanine aminotransferase; aspartate aminotransferase; potassium; sodium/potassium ratio; lactate; amylase; creatinine; total protein; phosphorous; sodium; zinc; and ammonia.

36. The method according to claim 2, wherein the method comprises analysing a sample collected from said population of fish to determine the amount of lactate dehydrogenase and creatine kinase as well as anywhere from none to one or more further analytes selected from the group consisting of: creatine kinase-MB; alanine aminotransferase; aspartate aminotransferase; potassium; sodium/potassium ratio; lactate; amylase; creatinine; total protein; phosphorous; sodium; zinc; and ammonia.

37. The method according to claim 2, wherein the method comprises analysing a sample collected from said population of fish to determine the amount of lactate dehydrogenase, creatine kinase and creatine kinase-MB as well as anywhere from none to one or more further analytes selected from the group consisting of: alanine aminotransferase; aspartate aminotransferase; potassium; sodium/potassium ratio; lactate; amylase; creatinine; total protein; phosphorous; sodium; zinc; and ammonia.

38. The method according to claim 2, wherein the method comprises analysing a sample collected from said population of fish to determine the amount of lactate dehydrogenase, creatine kinase, creatine kinase-MB, and alanine aminotransferase as well as anywhere from none to one or more further analytes selected from the group consisting of: aspartate aminotransferase; potassium; sodium/potassium ratio; lactate; amylase; creatinine; total protein; phosphorous; sodium; zinc; and ammonia.

39. The method according to claim 2, wherein the method comprises analysing a sample collected from said population of fish to determine the amount of lactate dehydrogenase, creatine kinase, creatine kinase-MB, alanine aminotransferase, and aspartate aminotransferase as well as anywhere from none to one or more further analytes selected from the group consisting of: potassium; sodium/potassium ratio; lactate; amylase; creatinine; total protein; phosphorous; sodium; zinc; and ammonia.

40. The method according to claim 2, wherein the method comprises analysing a sample collected from said population of fish to determine the amount of lactate dehydrogenase, creatine kinase, creatine kinase-MB, alanine aminotransferase, and aspartate aminotransferase as well as anywhere from none to one or more further analytes selected from the group consisting of: potassium; sodium/potassium ratio; lactate; amylase; creatinine; total protein; phosphorous; sodium; zinc; and ammonia.

41. The method according to claim 2, wherein the method comprises analysing a sample collected from said population of fish to determine the amount of lactate dehydrogenase, creatine kinase, creatine kinase-MB, alanine aminotransferase, aspartate aminotransferase and potassium as well as anywhere from none to one or more further analytes selected from the group consisting of: sodium/potassium ratio; lactate; amylase; creatinine; total protein; phosphorous; sodium; zinc; and ammonia.

42. The method according to claim 2, wherein the method comprises analysing a sample collected from said population of fish to determine the amount of lactate dehydrogenase, creatine kinase, creatine kinase-MB, alanine aminotransferase, aspartate aminotransferase and potassium.
